(19) United States
(12) Patent Application Publication
(10) Pub. No.: US 2004/0082555 A1
(43) Pub. Date: Apr. 29, 2004

(54) Use of gaba, inverse agonists in combination with nicotine receptor partial agonist, estrogen, selective estrogen modulators, or vitamin E for the treatment of cognitive disorders

(75) Inventor: Anabella Villalobos, Niantic, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 10/727,934

(22) Filed: Dec. 2, 2003

Related U.S. Application Data
(63) Continuation of application No. 10/083,743, filed on Feb. 26, 2002.
(60) Provisional application No. 60/272,566, filed on Mar. 1, 2001.

(51) Int. Cl. A61K 31/56; A61K 31/355

(52) U.S. Cl. 514/171; 514/458

(57) ABSTRACT

A pharmaceutical composition and method of treatment of diseases of cognitive dysfunction in a mammal comprising administration of a GABA_{A} inverse agonist or a pharmaceutically acceptable salt thereof; and a nicotine receptor partial agonist, an estrogenic agent, selective estrogen receptor modulator or vitamin E or a pharmaceutically acceptable salt thereof; and a pharmaceutically acceptable carrier. The GABA_{A} inverse agonist, and nicotine receptor partial agonist, estrogen, selective estrogen receptor modulator or vitamin E are present in amounts that render the composition effective enhancing cognition or in the treatment of diseases of cognitive dysfunction including but not limited to Alzheimer's Disease (AD), mild cognitive impairment, age-related cognitive decline, vascular dementia, Parkinson's disease, Huntington's disease, memory impairment associated with depresssion or anxiety, schizophrenia, Down's syndrome, stroke, traumatic brain injury (TBI), AIDS associated dementia and attention deficit disorder. The method of using these compositions is also disclosed.

This application claims priority from U.S. provisional application Serial No. 60/272,566, filed Mar. 1, 2001, which is incorporated herein by reference in its entirety.

### BACKGROUND OF THE INVENTION

The present invention relates to pharmaceutical compositions for the prevention and/or treatment of diseases of cognitive dysfunction in a mammal comprising GABA_{A} inverse agonists in combination with nicotine receptor partial agonists (NRPA), estrogen, selective estrogen receptor modulators (SERMS) or vitamin E and a pharmaceutically acceptable carrier. The pharmaceutical compositions are useful in enhancing memory in patients suffering from diseases of cognitive dysfunction such as, but not limited to, Alzheimer's Disease (AD), mild cognitive impairment, age-related cognitive decline, vascular dementia, Parkinson's disease, Huntington's disease, memory impairment associated with depression or anxiety, schizophrenia, Down's syndrome, stroke, traumatic brain injury (TBI), AIDS associated dementia and attention deficit disorder.

Cognitive and/or degenerative brain disorders are characterized clinically by progressive loss of memory, cognition, reasoning, judgment and emotional stability that gradually leads to profound mental deterioration and ultimately death. In an example of such disorders, AD is a common cause of progressive mental failure (dementia) in aged humans and is believed to represent the fourth most common medical cause of death in the United States. In particular, AD is associated with degeneration of cholinergic neurons in the basal forebrain that play a fundamental role in cognitive functions, including memory [Becker et al., Drug Development Research, 12, 163-195 (1988)]. Cognitive and/or degenerative brain disorders have been observed in varied races and ethnic groups worldwide and presents a major public health problem. These diseases are currently estimated to affect about two to three million individuals in the United States alone. These diseases are incurable with presently used medications and will increase worldwide as the human life span increases.

Modulators of the GABA_{A} receptors are capable of enhancing cognition in rodent models. In such models, it has been demonstrated that a selective inverse agonist profile can lead to cognitive enhancers devoid of or with minimum proconvulsant, anxiogenic and stimulant activity. The GABA_{A} inverse agonist binding and functional profile is described below:

TABLE 1

| Binding | Oocyte Functional Profile | | | |
|---|---|---|---|---|
| Ki | α1β2γ2 | α2β3γ2 | α3β3γ2 | α5β3γ2 |
| Ro15-1788 | EC₅₀/ | EC₅₀/ | EC₅₀/ | EC₅₀/ |
| Rat cortex | Efficacy | Efficacy | Efficacy | Efficacy |
| 100 nM, | 200 nM, | Any*/>10% | Any*/>10% | 200 nM, |
| preferably | preferably | | | preferably |
| <30 nM | <150 nM/ | | | <150 nM/ |
| | <−10% or >+10% | | | <−10% |
| *Though a wide range of EC₅₀ values at the α2β3γ2 and α3β3γ2 subtype receptors is permitted, in practice the “Any/>10%” criteria are used for compounds having EC₅₀ values at these subtypes below or equal to 100 times the EC₅₀ values at the α1β2γ2 and α5β3γ2 subtype receptors. | | | | |
| # When the EC₅₀ value of the compound at the α2β3γ2 and α3β3γ2 subtype receptor is more than 100 times greater than at the α1β2γ2 and α5β3γ2 subtype receptors then <10% in vitro efficacy would be acceptable. | | | | |

A compound is identified as having cognitive enhancing potential when the EC₅₀ value of the compound at the α1β2γ2 and/or α5β3γ2 subtype receptors is less than 200 nM, preferably less than 150 nM, and the efficacy measured is less than −5% or preferably less than −10%, and the efficacy measured at the α2β3γ2 and α3β3γ2 subtype receptors is greater than 5% or preferably greater than 10%.

The term NRPA refers to all chemical compounds which bind at neuronal nicotinic acetylcholine specific receptor sites in mammalian tissue and elicit a partial agonist response. A partial agonist response is defined here to mean a partial, or incomplete functional effect in a given functional assay. Additionally, a partial agonist will also exhibit some degree of antagonist activity by its ability to block the action of a full agonist (Feldman, R. S., Meyer, J. S. & Quenzer, L. F. Principles of Neuropsychopharmacology, 1997; Sinauer Assoc. Inc.).

NRPAs are expected to improve cognitive function in the above mentioned conditions. Referenced herein are well-documented findings that cholinergic mechanisms are important for normal cognitive functioning and that cholinergic hypofunction accompanies the cognitive deficits associated with Alzheimer's Disease (AD). It has been shown previously that nicotine administration improves some aspects of cognitive performance in both animal models of cognitive function and in patients with AD [Wilson et al., Pharmacology Biochemistry and Behavior, 51,509-514 (1995); Americ et al., Alzheimer Disease and Associated Disorders, 9(suppl 2), 50-61 (1995); Buccafusco et al., Behavioural Pharmacology, 10, 68190 (1999)].

The present invention also relates to the combination use of GABA_{A} inverse agonists and NRPAs, which result in cognition enhancement. As described above, modulators of the GABA_{A} receptors are capable of enhancing cognition in rodent models of cognition. In such models, it has been demonstrated that a selective inverse agonist profile (such as the one described in Table 1) can lead to cognitive enhancers devoid of or with minimum proconvulsant, anxiogenic and stimulant activity.

The present invention also relates to the combination use of GABA_{A} inverse agonists and estrogen and/or selective estrogen receptor modulators (SERMs) which result in cognition enhancement. Estrogen has been shown to have protective effects in both in vivo model systems of cognitive dysfunction as well as human clinical studies. Singh et al. [Brain Research, 644, 305-312 (1994)] demonstrates a decline of cognitive function in the ovarectomized rat which can be prevented by administration of estrogen. Fifteen clinical studies examining the role of estrogen replacement therapy in cognition demonstrate statistically significant improvements in cognitive function [Haskell et al., Journal of Clinical Epidemiology, 50(11), 1249-1264. (1997)].

The present invention also relates to the combination use of GABA_{A} inverse agonists and vitamin E, which result in cognition enhancement. Vitamin E displays anti-oxidant properties and as such it is expected to display neuroprotective activity.

It is expected that combinations of GABA_{A} inverse agonists with these agents would be useful in the treatment of disorders associated with cognition impairment including, but not limited to, Alzheimer's Disease (AD), mild cognitive impairment, age-related cognitive decline, vascular dementia, Parkinson's disease, Huntington's disease, memory impairment associated with depresssion or anxiety, schizophrenia, Down's syndrome, stroke, traumatic brain injury (TBI), AIDS associated dementia and attention deficit disorder.

### SUMMARY OF THE INVENTION

This invention provides a combination of a GABA_{A} inverse agonist and a nicotine receptor partial agonist, an estrogenic agent, a selective estrogen receptor modulator (SERM), or vitamin E for separate, sequential or simultaneous administration.

This invention also provides a combination of a nicotine receptor partial agonist, an estrogenic agent, a selective estrogen receptor modulator (SERM), or vitamin E and an inverse agonist of the GABA_{A} α5 receptor wherein the inverse agonist has a functional efficacy at the α5 receptor subtype of less than 20%, and a functional efficacy at the α₁, α₂ and α₃ receptor subtypes of between −20 and +20%.

This invention also provides a combination of a nicotine receptor partial agonist, an estrogenic agent, a selective estrogen receptor modulator (SERM), or vitamin E and a GABA_{A} inverse agonist wherein the inverse agonist has a functional efficacy at the α1 and/or α5 receptor subtypes of less than −5%, preferably less than −10%, and the efficacy measured at the α2 and α3 receptor subtypes is greater than 5% or preferably greater than 10%.

This invention also provides a combination of a nicotine receptor partial agonist, an estrogenic agent, a selective estrogen receptor modulator (SERM), or vitamin E and a GABA_{A} inverse agonist wherein the inverse agonist has functional potency (EC50 values) at the α1 and/or α5 receptor subtypes of 200 nM, preferably less than 150 nM.

This invention also provides a combination of a nicotine receptor partial agonist, an estrogenic agent, a selective estrogen receptor modulator (SERM), or vitamin E and an inverse agonist of the GABA_{A} α5 receptor wherein the inverse agonist has a functional efficacy at the α5 receptor subtype of less than −5%, preferably less than −10%, and the efficacy measured at the α1, α2 and α3 receptor subtypes is greater than 5% or preferably greater than 10%.

This invention also provides a combination of a nicotine receptor partial agonist, an estrogenic agent, a selective estrogen receptor modulator (SERM), or vitamin E and an inverse agonist of the GABA_{A} α5 receptor wherein the inverse agonist has a functional potency (EC50 values) at the α5 receptor subtype of 200 nM, preferably less than 150 nM.

This invention also provides a combination of a nicotine receptor partial agonist, an estrogenic agent, a selective estrogen receptor modulator (SERM), or vitamin E and a GABA_{A} inverse agonist wherein the inverse agonist at the α1 and/or α5 receptor subtypes have a binding Ki of 100 nM, preferably less than 30 nM.

Another aspect of this invention is a method of enhancing cognition or the treatment of a disorder involving cognitive dysfunction in a mammal comprising administering to the mammal, an amount of (a) a GABA_{A} inverse agonist or a pharmaceutically acceptable salt thereof; and (b) a nicotine receptor partial agonist, an estrogenic agent, a selective estrogen receptor modulator (SERM), or vitamin E or a pharmaceutically acceptable salt thereof; wherein the active ingredients (a) and (b) are administered in amounts that render the combination of the two ingredients effective in cognition or the enhancement of a disorder involving treatment of disorders cognitive dysfunction.

A method of treating a disorder or condition selected from the group consisting of, Alzheimer's Disease (AD), mild cognitive impairment, age-related cognitive decline, vascular dementia, Parkinson's disease, Huntington's disease, memory impairment associated with depresssion or anxiety, schizophrenia, Down's syndrome, stroke, traumatic brain injury (TBI), AIDS associated dementia and attention deficit disorder comprises administering to a mammal (a) a GABA_{A} inverse agonist or a pharmaceutically acceptable salt thereof; and (b) a nicotine receptor partial agonist, an estrogenic agent, a selective estrogen receptor modulator (SERM), or vitamin E or a pharmaceutically acceptable salt thereof; where in the active agents (a) and (b) above are administered in amounts that render the combination of the two ingredients effective in treating, Alzheimer's Disease (AD), mild cognitive impairment, age-related cognitive decline, vascular dementia, Parkinson's disease, Huntington's disease, memory impairment associated with depresssion or anxiety, schizophrenia, Down's syndrome, stroke, traumatic brain injury (TBI), AIDS associated dementia and attention deficit disorder.

This invention also provides a method for treating cognitive disorders in a mammal which comprises administering to a mammal in need of such treatment an effective amount of a composition comprising a GABA_{A} inverse agonist in combination with a nicotine receptor partial agonist, an estrogenic agent, a selective estrogen receptor modulator (SERM), or vitamin E wherein said GABA_{A} inverse agonist is selected from a compound of Formula I below.

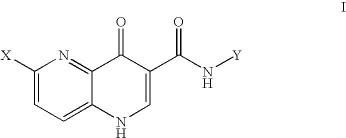

wherein:

X is hydrogen, halogen, —OR₁, NR₂R₃, C₁-C₆ alkyl optionally substituted with up to three groups selected independently from halogen and hydroxy, or —NR₂R₃; or X is phenyl, naphthyl, 1-(5,6,7,8-tetrahydro)naphthyl or 4-(1,2-dihydro)indenyl, pyridinyl, pyrimidyl, isoquinolinyl, 1,2,3,4-tetrahydroisoquinolinyl, benzofuranyl, benzothienyl, each of which is optionally substituted with up to three groups selected from halogen, C₁-C₆ alkyl, C₁-C₄ alkoxy, C₁-C₆ alkylthio, hydroxy, amino, mono or di(C₁-C₆) alkylamino, cyano, nitro, trifluoromethyl; or

X represents a carbocyclic group (“the X carbocyclic group”) containing from 3-7 members, up to two of which are optionally hetero atoms selected from oxygen and nitrogen, where the X carbocyclic group is optionally substituted with one or more groups selected from halogen, (C₁-C₆)alkoxy, mono- or di(C₁-C₆)alkylamino, sulfonamide, aza(C₃-C₇)cycloalkyl, (C₃-C₇)cycloalkylthio, (C₁-C₆)alkylthio, phenylthio, or a heterocyclic group; and

Y is lower alkyl having 1-8 carbon atoms optionally substituted with up to two groups selected from halogen, (C₁-C₆)alkoxy, mono- or di(C₁-C₆)alkylamino, sulfonamide, aza(C₃-C₇)cycloalkyl, (C₃-C₇)cycloalkylthio, (C₁-C₆)alkylthio, phenylthio, a heterocyclic group, —OR₄, —NR₅R₆, SR₇, or aryl; or

Y is a carbocyclic group (“the Y carbocyclic group”) having from 3 to 7 members, where up to three of which are optionally hetero atoms selected from oxygen and nitrogen and where any member of the Y carbocyclic group is optionally substituted with halogen, —OR₄, —NR₅R₆, SR₇, aryl or a heterocyclic group; and

R₁ is hydrogen, lower alkyl having 1-6 carbon atoms, or cycloalkyl having 3-7 carbon atoms, where each alkyl may be optionally substituted with —OR₄ or —NR₅R₆;

R₂ and R₃ are the same or different and represent hydrogen, lower alkyl optionally mono- or disubstituted with alkyl, aryl, halogen, or mono- or di-lower alkyl; aryl or aryl (C₁-C₆)alkyl where each aryl is optionally substituted with up to three groups selected from halogen, hydroxy, C₁-C₆ alkyl, C₁-C₆ alkoxy, or mono- or di(C₁-C₆)alkylamino;

cycloalkyl having 3-7 carbon atoms optionally mono or disubstituted with halogen, alkoxy, or mono- or di-lower alkyl; or —SO₂R₆;

R₄ is as defined for R₁;

R₅ and R₆ carry the same definitions as R₂ and R₃, respectively;

R₇ is hydrogen, lower alkyl having 1-6 carbon atoms, or cycloalkyl having 3-7 atoms; and

R₈ is lower alkyl having 1-6 carbon atoms, cycloalkyl having 3-7 carbon atoms, or optionally substituted phenyl; and

said cognitive disorder is selected from Alzheimer's Disease (AD), mild cognitive impairment, age-related cognitive decline, vascular dementia, Parkinson's disease, Huntington's disease, memory impairment associated with depresssion or anxiety, schizophrenia, Down's syndrome, stroke, traumatic brain injury (TBI), AIDS associated dementia and attention deficit disorder; and

This invention also provides a method for treating cognitive disorders in a mammal which comprises administering to a mammal in need of such treatment an effective amount of a composition comprising a GABA inverse agonist in combination with a nicotine receptor partial agonist, an estrogenic agent, a selective estrogen receptor modulator (SERM), or vitamin E wherein said GABA_{A} inverse agonist is selected from the group consisting of:

N-n-Butyl-6-chloro-4-oxo-1,4-tetrahydro-1,5-naphthyridine-3-carboxamide;

N-n-Butyl-6-ethoxy-4-oxo-1,4-tetrahydro-1,5-naphthyridine-3-carboxamide;

N-(2-Ethylthio)ethyl-6-methoxy-4-oxo-1,4-tetrahydro-1,5-naphthyridine-3-carboxamide;

N-n-Pentyl-6-ethoxy-4-oxo-1,4-tetrahydro-1,5-naphthyridine-3-carboxamide;

N-Benzyl-6-ethoxy-4-oxo-1,4-tetrahydro-1,5-naphthyridine-3-carboxamide;

N-(2-Tetrahydrofuranyl)methyl-6-ethoxy-4-oxo-1,4-tetrahydro-1,5-naphthyridine-3-carboxamide;

N-Isoamyl-6-ethoxy-4-oxo-1,4-tetrahydro-1,5-naphthyridine-3-carboxamide;

N-(3-Methoxybenzyl)-6-ethoxy-4-oxo-1,4 tetrahydro-1,5-naphthyridine-3-carboxamide;

N-(3-Ethoxy)propyl-6-ethoxy-4-oxo-1,4-tetrahydro-1,5-naphthyridine-3-carboxamide;

N-2-(2-Methyl)butyl-6-ethoxy-4-oxo-1,4-tetrahydro-1,5-naphthyndine-3 carboxamide;

N-5-Pentanol-6-ethoxy-4-oxo-1,4-tetrahydro-1,5-naphthyridine-3-carboxamide;

N-Benzyl-6-methoxy-4-oxo-1,4-tetrahydro-1,5-naphthyridine-3-carboxamide;

N-(2-Fluorobenzyl)-6-methoxy-4-oxo-1,4-tetrahydro-1,5-naphthyridine-3-carboxamide;

N-(3-Fluorobenzyl)-6-methoxy-4-oxo-1,4-tetrahydro-1,5-naphthyridine-3-carboxamide;

N-(4-Fluorobenzyl)-6-methoxy-oxo-1,4-tetrahydro-1,5-naphthyridine-3-carboxamide;

N-(4/5-Imidazolyl)methyl-6-ethoxy-4-oxo-1,4-tetrahydro-1,5-naphthyridine-3-carboxamide;

N-(3-Thienyl)methyl-6-ethoxy-4-oxo-1,4-tetrahydro-1,5-naphthyridine-3-carboxamide;

N-(2-Tetrahydropyranyl)methyl-6-ethoxy-4-oxo-1,4-tetrahydro-1,5-naphthyridine-3-carboxamide;

N-(2-Fluorobenzyl)6-ethoxy-4-oxo-1,4-tetrahydro-1,5-naphthyridine-3-carboxamide;

N-(3,5-Fluorobenzyl)-6-ethoxy-4-oxo-1,4-tetrahydro-1,5-naphthyridine-3-carboxamide;

N-(4-Fluorobenzyl)-6-ethoxy-4-oxo-1,4-tetrahydro-1,5-naphthyridine-3-carboxamide;

N-(4-Methoxybenzyl)-6-ethoxy-4-oxo-1,4-tetrahydro-1,5-naphthyridine-3-carboxamide;

N-(4-Methylbenzyl)-6-ethoxy-4-oxo-1,4-tetrahydro-1,5-naphthyridine-3-carboxamide;

N-(2-Thienyl)methyl-6-(2-methoxyethoxy)-4-oxo-1,4-tetrahydro-1,5-naphthyridine-3-carboxamide;

N-(2-Thienyl)methyl-6-morpholino-4-oxo-1,4-tetrahydro-1,5-naphthyridine-3-carboxamide;

N-(2-Thienyl)methyl-6-dimethylamino-4-oxo-1,4-tetrahydro-1,5-naphthyridine-3-carboxamide;

N-(4-Methylaminomethyl)benzyl-6-ethoxy-4-oxo-1,4-tetrahydro-1,5-naphthyridine-3-carboxamide;

N-(3-Methylaminomethyl)benzyl-6-ethoxy-4-oxo-1,4-tetrahydro-1,5-naphthyridine-3-carboxamide hydrochloride; and

N-[4(Imidazolylmethy)lbenzyl-6-ethoxy-4-oxo-1,4-tetrahydro-1,5-naphthyridine-3-carboxamide.

This invention also provides a method for treating cognitive disorders in a mammal which comprises administering to a mammal in need of such treatment an effective amount of a composition comprising a GABA_{A} inverse agonist in combination with a nicotine receptor partial agonist, an estrogenic agent, a selective estrogen receptor modulator (SERM), or vitamin E wherein said GABA_{A} inverse agonist compound is selected from a compound which is

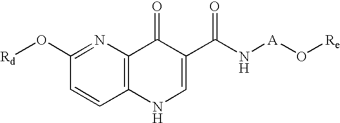

wherein

A is C₁-C₆ alkylene;

R_{d} and Rₑ are independently lower alkyl groups.

This invention also provides a method for treating cognitive disorders in a mammal which comprises administering to a mammal in need of such treatment an effective amount of a composition comprising a GABA_{A} inverse agonist in combination with a nicotine receptor partial agonist, an estrogenic agent, a selective estrogen receptor modulator (SERM), or vitamin E wherein said GABA_{A} inverse agonist is selected from a compound which is

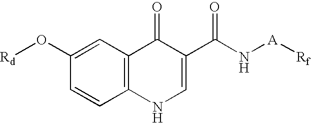

wherein

A is C₁-C₆ alkylene;

R_{d} is lower alkyl; and

R_{f} is a group of the formula:

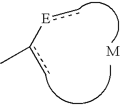

where E is oxygen or nitrogen; and

M is C₁-C₃ alkylene or nitrogen.

This invention also provides a method for treating cognitive disorders in a mammal which comprises administering to a mammal in need of such treatment an effective amount of a composition comprising a GABA_{A} inverse agonist in combination with a nicotine receptor partial agonist, an estrogenic agent, a selective estrogen receptor modulator (SERM), or vitamin E wherein said GABA_{A} inverse agonist is selected from a compound which is

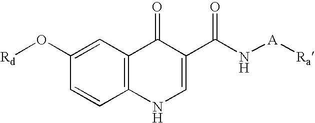

wherein

A is C₁-C₆ alkylene;

R_{d} is lower alkyl; and

Rₐ′ is phenyl optionally mono-, di- or trisubstituted with halogen, lower alkyl, lower alkoxy, or mono- or di-C₁-C₆ alkylamino, or mono-di-C₁-C₆ alkylamino lower alkyl; or

Rₑ′ is a heteroaryl group, that is, one or more aromatic ring systems of 5-,6- or 7-membered rings containing at least one and up to four hetero atoms selected from nitrogen, oxygen or sulfur. Such heteroaryl groups include, for example, thienyl, furanyl, thiazolyl, imidazolyl, (is)oxazolyl, pyridyl, pyrimidinyl, (iso)quinolinyl, naphthyridinyl, benzimidazolyl, and benzoxazolyl.

This invention also provides a method for treating cognitive disorders in a mammal which comprises administering to a mammal in need of such treatment an effective amount of a composition comprising a GABA_{A} inverse agonist in combination with a nicotine receptor partial agonist, an estrogenic agent, a selective estrogen receptor modulator (SERM), or vitamin E wherein said GABA_{A} inverse agonist compound is selected from a compound which is

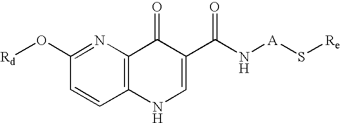

wherein

A is C₁-C₆ alkylene; and

R_{d} and Rₑ are independently lower alkyl groups.

This invention also provides a method for treating cognitive disorders in a mammal which comprises administering to a mammal in need of such treatment an effective amount of a composition comprising a GABA_{A} inverse agonist in combination with a nicotine receptor partial agonist, an estrogenic agent, a selective estrogen receptor modulator (SERM), or vitamin E wherein said GABA_{A} inverse agonist is selected from a compound which is

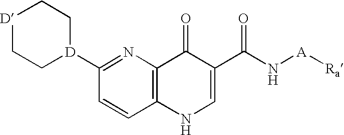

wherein

D is nitrogen or CH;

D′ is —NH; —N lower alkyl

A is C₁-C₆ alkylene; and

Rₐ′ is phenyl optionally mono-, di- or trisubstituted with halogen, lower alkyl, lower alkoxy, or mono- or di-C₁-C₆ alkylamino, or mono- or di-C₁-C₆alkylamino lower alkyl.

This invention also provides a method for treating cognitive disorders in a mammal which comprises administering to a mammal in need of such treatment an effective amount of a composition comprising a GABA_{A} inverse agonist in combination with a nicotine receptor partial agonist, an estrogenic agent, a selective estrogen receptor modulator (SERM), or vitamin E wherein said GABA_{A} inverse agonist is selected from a compound which is

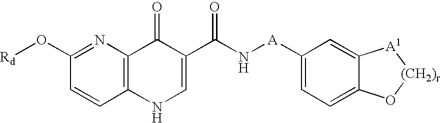

wherein

A is C₁-C₆ alkylene; and

R_{d} is lower alkyl;

A′ represents oxygen or methylene; and

r is an integer of from 1-3.

This invention also provides a method for treating cognitive disorders in a mammal which comprises administering to a mammal in need of such treatment an effective amount of a composition comprising a GABA_{A} inverse agonist in combination with a nicotine receptor partial agonist, an estrogenic agent, a selective estrogen receptor modulator (SERM), or vitamin E wherein said GABA_{A} inverse agonist is selected from a compound which is

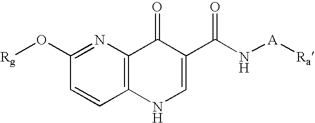

wherein

A is C₁-C₆ alkylene;

R_{g} is lower alkyloxy lower alkyl; and

Rₐ′ is phenyl optionally mono-, di-, or trisubstituted with halogen, lower alkyl, lower alkoxy, or mono- or di-C₁-C₆ alkylamino, or mono- or di-C₁-C₆ alkylamino lower alkyl.

This invention also provides a method for treating cognitive disorders in a mammal which comprises administering to a mammal in need of such treatment an effective amount of a composition comprising a GABA_{A} inverse agonist in combination with a nicotine receptor partial agonist, an estrogenic agent, a selective estrogen receptor modulator (SERM), or vitamin E wherein said GABA_{A} inverse agonist is selected from a compound which is

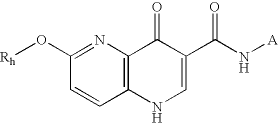

wherein

A is lower alkyl having 1-8 carbon atoms or cycloalkyl having 3-7 carbon atoms, any of which may be optionally substituted with one or more hydroxy groups and Rₕ is lower alkyl.

This invention also provides a method for treating cognitive disorders in a mammal which comprises administering to a mammal in need of such treatment an effective amount of a composition comprising a GABA_{A} inverse agonist in combination a nicotine receptor partial agonist, an estrogenic agent, a selective estrogen receptor modulator (SERM), or vitamin E wherein said cognitive disorder is selected from Alzheimer's Disease (AD), mild cognitive impairment, age-related cognitive decline, vascular dementia, Parkinson's disease, Huntington's disease, memory impairment associated with depresssion or anxiety, schizophrenia, Down's syndrome, stroke, traumatic brain injury (TBI), AIDS associated dementia and attention deficit disorder.

This invention also provides a method for treating cognitive disorders in a mammal which comprises administering to a mammal in need of such treatment an effective amount of a composition comprising a GABA_{A} inverse agonist in combination with a nicotine receptor partial agonist, an estrogenic agent, a selective estrogen receptor modulator (SERM), or vitamin E wherein said cognitive disorder is Alzheimer's disease.

This invention also provides a method for treating cognitive disorders in a mammal which comprises administering to a mammal in need of such treatment an effective amount of a composition comprising a GABA_{A} inverse agonist in combination with a nicotine receptor partial agonist, an estrogenic agent, a selective estrogen receptor modulator (SERM), or vitamin E wherein said cognitive disorder is mild cognitive impairment.

This invention also provides a method for treating cognitive disorders in a mammal which comprises administering to a mammal in need of such treatment an effective amount of a composition comprising a GABA_{A} inverse agonist in combination with a nicotine receptor partial agonist, an estrogenic agent, a selective estrogen receptor modulator (SERM), or vitamin E in which said nicotine receptor partial agonists are selected from:

9-bromo-1,2,3,4,5,6-hexahydro-1,5-methano-pyrido[1,2-a][1,5]diazocin-8-one;

9-chloro-1,2,3,4,5,6-hexahydro-1,5-methano-pyrido[1,2-a][1,5]diazocin-8-one;

9-fluoro-1,2,3,4,5,6-hexahydro-1,5-methano-pyrido[1,2-a][1,5]diazocin-8-one;

9-ethyl-1,2,3,4,5,6-hexahydro-1,5-methano-pyrido[1,2-a][1.5]diazocin-8-one;

9-methyl-1,2,3,4,5,6-hexahydro-1,5-methano-pyrido[1,2-a][1,5]diazocin-8-one;

9-phenyl-1,2,3,4,5,6-hexahydro-1,5-methano-pyrido[1,2-a][1,5]diazocin-8-one;

9-vinyl-1,2,3,4,5,6-hexahydro-1,5-methano-pyrido[1,2-a][1,5]diazocin-8-one;

9-bromo-3-methyl-1,2,3,4,5,6-hexahydro-1,5-methano-pyrido[1,2a][1,5]diazocin-8-one;

3-benzyl-9-bromo-1,2,3,4,5,6-hexahydro-1,5-methano-pyrido[1,2-a][1,5]diazocin-8-one;

3-benzyl-9-chloro-1,2,3,4,5,6-hexahydro-1,5-methano-pyrido[1,2-a][1,5]diazocin-8-one;

9-acetyl-1,2,3,4,5,6-hexahydro-1,5-methano-pyrido[1,2a][1,5]diazocin-8-one;

9-iodo-1,2,3,4,5,6-hexahydro-1,5-methano-pyrido[1,2a][1,5]diazocin-8-one;

9-cyano-1,2,3,4,5,6-hexahydro-1,5-methano-pyrido[1,2a][1,5]diazocin-8-one;

9-ethynyl-1,2,3,4,5,6-hexahydro-1,5-methano-pyrido[1,2a][1,5]diazocin-8-one;

9-(2-propenyl)-1,2,3,4,5,6-hexahydro-1,5-methano-pyrido[1,2a][1,5]diazocin-8-one;

9-(2-propyl)-1,2,3,4,5,6-hexahydro-1,5-methano-pyrido[1,2a][1,5]diazocin-8-one;

9-carbomethoxy-1,2,3,4,5,6-hexahydro-1,5-methano-pyrido[1,2a][1,5]diazocin-8-one;

9-carboxyaldehyde-1,2,3,4,5,6-hexahydro-1,5-methano-pyrido[1,2a][1,5]diazocin-8-one;

9-(2,6-difluorophenyl)1,2,3,4,5,6-hexahydro-1,5-methano-pyrido-[1,2a][1,5]diazocin-8-one;

9-phenyl-1,2,3,4,5,6-hexahydro-1,5-methano-pyrido[1,2a][1,5]diazocinone;

9-(2-fluorophenyl)-1,2,3,4,5,6-hexahydro-1,5-methano-pyrido[1,2a][1,5]diazocin-8-one;

9-(4-fluorophenyl)1,2,3,4,5,6-hexahydro-1,5-methano-pyrido[1,2a][1,5]diazocin-8-one;

9-(3-fluorophenyl)1,2,3,4,5,6-hexahydro-1,5-methano-pyrido[1,2a][1,5]diazocin-8-one;

9-(3,5-difluorophenyl)-1,2,3,4,5,6-hexahydro-1,5-methano-pyrido[1,2a][1,5]diazocin-8-one;

9-(2,4-difluorophenyl)-1,2,3,4,5,6-hexahydro-1,5-methano-pyrido[1,2a][1,5]diazocin-8-one;

9-(2,5-difluorophenyl)-1,2,3,4,5,6-hexahydro-1,5-methano-pyrido[1,2a][1.5]diazocin-8-one;

6-methyl-5-oxo-6, 13-diazatetracyclo[9.3.1.0^{2,10}.0^{4,8}]pentadeca-2(10),3,8-triene;

5-oxo-6,13-diazatetracyclo[9.3.1.0_{2,10}.0^{4,8}]pentadeca-2(10),3,8-triene;

6-oxo-5,7,13-triazatetracyclo[9.3.1.0^{2,10}.0^{4,8}]pentadeca-2(10),3,8-triene;

4,5-difluoro-10-aza-tricyclo[6.3.1.0^{2,7}]dodeca-2(7),3,5-triene;

5-fluoro-10-aza-tricyclo[6.3.1.0^{2,7}]dodeca-2(7),3,5-triene-4-carbonitrile;

4-ethynyl-5-fluoro-10-aza-tricyclo[6.3.1.0^{2,7}]dodeca-2(7),3,5-triene;

5-ethynyl-10-aza-tricyclo[6.3.1.0^{2,7}]dodeca-2(7),3,5-triene-4-carbonitrile;

6-methyl-5-thia-5-dioxa-6,13-diazatetracyclo[9.3.1.0^{2,10}.0^{4,8}]pentadeca-2(10),3,8-triene;

10-aza-tricyclo[6.3.1.0^{2,7}]dodeca-2(7),3,5-triene;

4-fluoro-10-aza-tricyclo[6.3.1.0^{2,7}]dodeca-2(7),3,5-triene;

4-methyl-10-aza-tricyclo[6.3.1.0^{2,7}]dodeca-2(7),3,5-triene;

4-trifluoromethyl-10-aza-tricyclo[6.3.1.0^{2,7}]dodeca-2(7),3,5-triene;

4-nitro-10-azatricyclo[6.3.1.0_{2,7}]dodeca-2(7),3,5-triene;

7-methyl-5,7,13-triazatetracyclo[9.3.1.0^{2,10}.0^{4,8}]pentadeca-2(10),3,5,8-tetraene;

6-methyl-5,7,13-triazatetracyclo[9.3.1.0^{2,10}.0^{4,8}]pentadeca-2(10),3,5,8-tetraene;

6,7-dimethyl-5,7,13-triazatetracyclo[9.3.1.0^{2,10}.0^{4,8}]pentadeca-2(10),3,5,8-tetraene;

6-methyl-7-phenyl-5,7,13-triazatetracyclo[9.3.1.0^{2,10}.0^{4,8}]pentadeca-2(10),3,5,8-tetraene;

6,7-dimethyl-5,8,14-triazatetracyclo[10.3.1.0^{2,11}.0^{4,9}]hexadeca-2(11),3,5,7,9-pentaene;

5,8,14-triazatetracyclo[10.3.1.0^{2,10}0^{4,8}]hexadeca-2(11),3,5,7,9-pentaene;

14-methyl-5,8,14-triazatetracyclo[10.3.1.0^{2,11}.0^{4,9}]hexadeca-2(11),3,5,7,9-pentaene;

5-oxa-7,13-diazatetracyclo[9.3.1.0^{2,10}.0^{4,8}]pentadeca-2(10),3,6,8-tetraene;

6-methyl-5-oxa-7,13-diazatetracyclo[9.3.1.0^{2,10}.0^{4,8}]pentadeca-2(10),3,6,8-tetraene;

4-chloro-10-azatricyclo[6.3.1.0^{2,7}]dodeca-2(7),3,5-triene;

10-azatricyclo[6.3.1.0^{2,7}]dodeca-2(7),3,5-trien-4-yl cyanide;

1-(10-azatricyclo[6.3.1.0^{2,7}]dodeca-2(7),3,5-trien-4-yl)-1-ethanone;

10-azatricyclo[6.3.1.0^{2,7}]dodeca-2(7),3,5-trien-4-ol;

7-methyl-5-oxa-6,13-diazatetracyclo[9.3.1.0^{2,10}.0^{4,8}]pentadeca-2,4(8),6,9-tetraene;

4,5-dichloro-10-azatricyclo[6.3.1.0^{2,7}]dodeca-2(7),3,5-triene;

11-azatricyclo[7.3.1.0^{2,7}]trideca-2(7),3,5-triene-5-carbonitrile;

1-[11-azatricyclo[7.3.1.0^{2,7}]trideca-2(7),3,5-trien-5-yl]-1-ethanone;

1-[11-azatricyclo[7.3.10^{2,7}]trideca-2(7),3,5-trien-5-yl]-1-propanone;

4-fluoro-11-azatricyclo[7.3.1.0^{2,7}]trideca-2(7),3,5-triene-5-carbonitrile;

5-fluoro-11-azatricyclo[7.3.1.0^{2,7}]trideca-2(7),3,5-triene-4-carbonitrile;

6-methyl-7-thia-5,14-diazatetracyclo[10.3.1.0^{2,10}.0^{4,8}]hexadeca-2(10),3,5,8-tetraene;

6-methyl-5,7,14-triazatetracyclo[10.3.1.0^{2,10}.0^{4,8}]hexadeca-2(10),3,5,8-tetraene;

6,7-dimethyl-5,7,14-triazatetracyclo[10.3.1.0^{2,10}.0^{4,8}]hexadeca-2(10),3,5,8-tetraene;

5,7,14-triazatetracyclo[10.3.1.0^{2,10}.0^{4,8}]hexadeca-2(10),3,5,8-tetraene;

5,6-dimethyl-5,7,14-triazatetracyclo[10.3.1.0^{2,10}.0^{4,8}]hexadeca-2(10),3,6,8-tetraene;

5-methyl-5,7,14-triazatetracyclo[10.3.1.0^{2,10}_{.0}^{4,8}]hexadeca-2(10),3,6,8-tetraene;

6-(trifluoromethyl)-7-thia-5,14-diazatetracyclo[10.3.1.0^{2,10}.0^{4,8}]hexadeca-2(10),3,5,8-tetraene;

5,8,15-triazatetracyclo[11.3.1.0^{2,11}.0^{4,9}]heptadeca-2(11),3,5,7,9-pentaene;

7-methyl-5,8,15-triazatetracyclo[11.3.1.0^{2,11}.0^{4,9}]heptadeca-2(11),3,5,7,9-pentaene;

6-methyl-5,8,15-triazatetracyclo[11.3.1.0^{2,11}.0^{4,9}]heptadeca-2(11),3,5,7,9-pentaene;

6,7-dimethyl-5,8,15-triazatetracyclo[11.3.1.0^{2,10}.0^{4,8}]heptadeca-2(11),3,5,7,9-pentaene;

7-oxa-5,14-diazatetracyclo[10.3.1.0^{2,10}.0^{4,8}]hexadeca-2(10),3,5,8-tetraene;

6-methyl-7-oxa-5,14-diazatetracyclo[10.3.1.0^{2,10}.0^{4,8}]hexadeca-2(10),3,5,8-tetraene;

5-methyl-7-oxa-6,14-diazatetracyclo[10.3.1.0^{2,10}.0^{4,8}]hexadeca-2(10),3,5,8-tetraene;

6-methyl-5-oxa-7,14-diazatetracyclo[10.3.1.0^{2,10}.0^{4,8}]hexadeca-2(10),3,6,8-tetraene;

7-methyl-5-oxa-6,14-diazatetracyclo[10.3.1.0^{2,10}.0^{4,8}]hexadeca-2(10),3,6,8-tetraene;

4,5-difluoro-11-azatricyclo[7.3.1.0^{2,7}]trideca-2(7),3,5-triene;

4-chloro-5-fluoro-11-azatricyclo[7.3.1.0^{2,7}]trideca-2(7),3,5-triene;

5-chloro-4-fluoro-11-azatricyclo[7.3.1.0^{2,7}]trideca-2(7),3,5-triene;

4-(1-ethynyl)-5-fluoro-11-azatricyclo[7.3.1.0^{2,7}]trideca-2(7),3,5-triene;

5-(1-ethynyl)4-fluoro-12]-azatricyclo[7.3.1.0^{2,7}]trideca-2(7),3,5-triene;

5,6-difluoro-11-aza-tricyclo[7.3.1.0^{2,7}]-trideca-2,4,6-triene;

6-trifluoromethyl-11-aza-tricyclo[7.3.1.0^{2,7}]trideca-2,4,6-triene;

6-methoxy-11-aza-tricyclo[7.3.1.0^{2,7}]trideca-2(7),3,5-triene;

11-aza-tricyclo[7.3.1.0^{2,7}]trideca-2(7),3,5-trien-6-ol;

6-fluoro-11-aza-tricyclo[7.3.1.0^{2,7}]trideca-2(7),3,5-triene;

11-aza-tricylo[7.3.1.0^{2,7}]trideca-2(7),3,5-trien-5-ol;

4-nitro-11-aza-tricyclo[7.3.1.0^{2,7}]trideca-2(7),3,5-triene;

5-nitro-11-aza-tricyclo[7.3.1.0^{2,7}]trideca-2(7),3,5-triene;

5-fluoro-11-aza-tricyclo[7.3.1.0^{2,7}]trideca-2(7),3,5-triene; and

6-hydroxy-5-methoxy-11-aza-tricyclo[7.3.1.0^{2,7}]trideca-2(7),3,5-triene and their pharmaceutically acceptable salts and their optical isomers.

Preferably, the nicotine receptor partial agonist is selected from:

9-bromo-1,2,3,4,5,6-hexahydron-1,5-methano-pyrido[1,2-a][1,5]diazocin-8-one;

9-chloro-1,2,3,4,5,6-hexahydro-1,5-methano-pyrido[1,2-a][1,5]diazocin-8-one;

9-fluoro-1,2,3,4,5,6-hexahydro-1,5-methano-pyrido[1,2-a][1,5]diazocin-8-one;

9-acetyl-1,2,3,4,5,6-hexahydro-1,5-methano-pyrido[1,2a][1,5]diazocin-8-one;

9-iodo-1,2,3,4,5,6-hexahydro-1,5-methano-pyrido[1,2a][1,5]diazocin-8-one;

9-cyano-1,2,3,4,5,6-hexahydro-1,5-methano-pyrido[1,2a][1,5]diazocin-8-one;

9-carbomethoxy-1,2,3,4,5,6-hexahydro-1,5-methano-pyrido[1,2a][1,5]diazocin-8-one;

9-carboxyaldehyde-1,2,3,4,5,6-hexahydro-1,5-methano-pyrido[1,2a][1,5]diazocin-8-one;

9(2,6-difluorophenyl)-1,2,3,4,5,6-hexahydro-1,5-methano-pyrido[1,2a][1,5]diazocin-8-one;

9-phenyl-1,2,3,4,5,6-hexahydro-1,5-methano-pyrido[1,2a][1,5]diazocin-8-one;

9-(2-fluorophenyl 1,2,3,4,5,6-hexahydro-1,5-methano-pyrido[1,2a][1,5]diazocin-8-one;

6-methyl-5-thia-5-dioxa-6,13-diazatetracyclo[9.3.1.0^{2,10}.0^{4,8}]pentadeca-2(10),3,8-triene;

4-fluoro-10-aza-tricyclo[6.3.1.0^{2,7}]dodeca-2(7),3,5-triene;

4-trifluoromethyl-10-aza-tricyclo[6.3.1.0^{2,7}]dodeca-2(7),3,5-triene;

4-nitro-10-azatricyclo[6.3.1.0^{2,7}]dodeca-2(7),3,5-triene;

6-methyl-5,7,13-triazatetracyclo[9.3.1.0^{2,10}.0^{4,8}]pentadeca-2(10),3,5,8-tetraene;

6,7-dimethyl-5,8,14-triazatetracyclo[10.3.1.0^{2,11}.0^{4,8}]hexadeca-2(11),3,5,7,9-pentaene;

5,8,14-triazatetracyclo[10.3.1.0^{2,11}.0^{4,9}]hexadeca-2(11),3,5,7,9-pentaene;

5-oxa-7,13-diazatetracyclo[9.3.1.0^{2,10}.0^{4,8}]pentadeca-2(10),3,6,8-tetraene;

6-methyl-5-oxa-7,13-diazatetracyclo[9.3.1.0^{2,10}.0^{4,8}]pentadeca-2(10),3,6,8-tetraen;

10-azatricyclo[6.3.1.0^{2,7}]dodeca-2(7),3,5-trien-4-yl cyanide;

1-(10-azatricyclo[6.3.1.0^{2,7}]dodeca-2(7),3,5-trien-4-yl)-1-ethanone;

11-azatricyclo[7.3.1.0^{2,7}]trideca-2(7),3,5-triene-5-carbonitrile;

1-[11-azatricyclo[7.3.1.0^{2,7}]trideca-2(7),3,5-trien-5-yl]-1-ethanone;

1-[11-azatricyclo[7.3.1.0^{2,7}]trideca-2(7),3,5-trien-5-yl]-1-propanone;

4-fluoro-11-azatricyclo[7.3.1.0^{2,7}]trideca-2(7),3,5-triene-5-carbonitrile;

5-fluoro-11-azatricyclo[7.3.1.0^{2,7}]trideca-2(7),3,5-triene-4-carbonitrile;

6-methyl-7-thia-5,14-diazatetracyclo[10.3.1.0^{2,10}.0^{4,8}]hexadeca-2(10),3,5,8-tetraene;

6-methyl-5,7,14-triazatetracyclo[10.3.1.0^{2,10}.0^{4,8}]hexadeca-2(10),3,5,8-tetraene;

6,7-dimethy-5,7,14-triazatetracyclo[10.3.1.0^{2,10}.0^{4,8}]hexadeca-2(10),3,5,8-tetraene;

6-methyl-7-oxa-5,14-diazatetracyclo[10.3.1.0^{2,10}.0^{4,8}]hexadeca-2(10),3,5,8-tetraene;

6-methyl-5-oxa-7,14-diazatetracyclo[10.3.1.0^{2,10}.0^{4,8}]hexadeca-2(10),3,6,8-tetraene;

5,6-difluoro-11-aza-tricyclo[7.3.1.0^{2,7}]trideca-2,4,6-triene;

6-trifluoromethyl-11-aza-tricyclo[7.3.1.0^{2,7}]trideca-2,4,6-triene;

6-methoxy-11-aza-tricyclo[7.3.1.0^{2,7}]trideca-2(7),3,5-triene;

6-fluoro-11-aza-tricyclo[7.3.1.0^{2,7}]trideca-2(7),3,5-triene; and

11-aza-tricyclo[7.3.1.0^{2,7}]trideca-2(7),3,5-trien-5-ol

and their pharmaceutically acceptable salts and their optical isomers.

The estrogenic agent is estradiol or a pharmaceutically acceptable form of estradiol.

The estrogen receptor modulators are selected from estrogen, lasofoxifene, droloxifene, tamoxifen and raloxifene (Evista).

The present invention relates to a pharmaceutical composition for the enhancement of cognition or the treatment of disorders involving cognitive dysfunction in a mammal comprising (a) A GABA_{A} inverse agonist with binding and functional properties such as the ones described in Table 1 or pharmaceutical acceptable salt thereof; (b) a nicotine receptor partial agonist (NRPA), an estrogenic agent, a selective estrogen receptor modulator (SERM) or vitamin E or a pharmaceutically acceptable salt thereof; and (c), a pharmaceutically acceptable carrier; wherein the active ingredients (a) and (b) above are present in amounts that render the composition effective in the enhancement of cognition or the treatment of disorders of cognitive dysfunction.

The present invention also provides kits comprising:

a) a first compound, said first compound being a compound of formula I, an isomer thereof, a prodrug of said compound or isomer, or a pharmaceutically acceptable salt of said compound, isomer or prodrug; and a pharmaceutically acceptable carrier, vehicle or diluent in a first unit dosage form;

b) a second compound, said second compound being selected from the group consisting of a nicotine receptor partial agonist (NRPA), an estrogenic agent, a selective estrogen receptor modulator (SERM) or vitamin E and a pharmaceutically acceptable carrier, vehicle or diluent in a second unit dosage form; and

c) a container for containing said first and second dosage forms wherein the amounts of said first and second compounds result in an enhanced therapeutic effect.

The pharmaceutical compositions are useful in the enhancement of cognition or the treatment of disorders involving cognitive dysfunction including but not limited to Alzheimer's Disease (AD), mild cognitive impairment, age-related cognitive decline, vascular dementia, Parkinson's disease, Huntington's disease, memory impairment associated with depresssion or anxiety, schizophrenia, Down's syndrome, stroke, traumatic brain injury (TBI), AIDS associated dementia and attention deficit disorder.

A preferred aspect of this method is wherein the GABA_{A} inverse agonist is in combination with nicotinic receptor partial agonists (NRPA) or a pharmaceutically acceptable form of the foregoing compounds.

Another preferred aspect of this method is wherein the GABA_{A} inverse agonist is in combination with an estrogenic agent or a pharmaceutically acceptable form of estrogen.

Another preferred aspect of this method is wherein the GABA_{A} inverse agonist is in combination with a SERM selected from lasofoxifene, droloxifene, tamoxifen and raloxifene (Evista) or a pharmaceutically acceptable salt of one of the foregoing compounds.

Another preferred aspect of this method is wherein the GABA_{A} inverse agonist is in combination with vitamin E or a pharmaceutically acceptable form of vitamin E.

Another preferred aspect of this method is wherein the GABA_{A} inverse agonist is administered substantially simultaneously with the NRPA, an estrogenic agent, SERM, or vitamin E.

The pharmaceutical composition is used for enhancing cognition or treating a disorder involving cognitive dysfunction, including but not limited to Alzheimer's Disease (AD), mild cognitive impairment, age-related cognitive decline, vascular dementia, Parkinson's disease, Huntington's disease, memory impairment associated with depresssion or anxiety, schizophrenia, Down's syndrome, stroke, traumatic brain injury (TBI), AIDS associated dementia and attention deficit disorder in a mammal, including a human. The method comprises administering to said mammal a cognitive dysfunction attenuating effective amount of the above pharmaceutical composition comprising (a) a GABA_{A} inverse agonist with binding and functional properties such as the ones described in Table 1 or pharmaceutical acceptable salt thereof; (b) a nicotine receptor partial agonist (NRPA), an estrogenic agent, a selective estrogen receptor modulator (SERM) or vitamin E or a pharmaceutically acceptable salt thereof. In the pharmaceutical composition (a) and (b) are present in amounts that render the composition effective in treating such disorders.

The term “treating”, “treat” or “treatment” as used herein includes preventive (e.g., prophylactic), disease-modifying, and palliative treatment.

The chemist of ordinary skill will recognize that certain compounds of this invention will contain one or more atoms, which may be in a particular stereochemical or geometric configuration, giving rise to stereoisomers, tautomers and configurational isomers. All such isomers and mixtures thereof are included in this invention. Hydrates of the compounds of this invention are also included.

The chemist of ordinary skill will recognize that certain combinations of heteroatom-containing substituents listed in this invention define compounds which will be less stable under physiological conditions (e.g. those containing acetal or aminal linkages). Accordingly, such compounds are less preferred.

### DETAILED DESCRIPTION OF THE INVENTION

The GABA_{A} ligands disclosed above may be prepared by the methods described in WO 99/10347 by Neurogen Corp., published Mar. 4, 1999, which is incorporated herein by reference.

By lower alkyl in the present invention is meant straight or branched chain alkyl groups having 1-6 carbon atoms, such as, for example, methyl, ethyl, propyl, isopropyl, n-butyl, sec-butyl, tert-butyl, pentyl, 2-pentyl, isopentyl, neopentyl, hexyl, 2-hexyl, 3-hexyl, and 3-methylpentyl.

By cycloalkyl in the present invention is meant cycloalkyl groups having 3-7 atoms, such as, for example, cyclopropyl, cyclobutyl, cyclopentyl, cyclohexyl, and cycloheptyl.

By aryl is meant an aromatic carbocyclic group having a single ring (e.g., phenyl), multiple rings (e.g., biphenyl), or multiple condensed rings in which at least one is aromatic, (e.g., 1,2,3,4-tetrahydronapthyl, naphthyl, anthryl, or phenanthryl), which is optionally mono-, di-, or trisubsituted with, e.g., halogen, lower alkyl, lower alkoxy, lower alkylthio, trifluoromethyl, lower acyloxy, aryl, heteroaryl, and hydroxy.

By lower alkoxy in the present invention is meant straight or branched chain alkoxy groups having 1-6 carbon atoms, such as, for example, methoxy, ethoxy, propoxy, isopropoxy, n-butoxy, secbutoxy, tertbutoxy, pentoxy, 2-pentyl, isopentoxy, neopentoxy, hexoxy, 2-hexoxy, 3-hexoxy, and 3-methylpentoxy.

By cycloalkoxy in the present invention is meant cycloalkylalkoxy groups having 3-7 carbon atoms where cycloalkyl is defined above.

By halogen in the present invention is meant fluorine, bromine, chlorine, and iodine.

By heteroaryl (aromatic heterocycle) in the present invention is meant one or more aromatic ring systems of 5-, 6-, or 7-membered rings containing at least one and up to four hetero atoms selected from nitrogen, oxygen, or sulfur. Such heteroaryl groups include, for example, thienyl, furanyl, thiazolyl, imidazolyl, (is)oxazolyl, pyridyl, pyrimidinyl, (iso)quinolinyl, naphthridinyl, benzimidazolyl, and benzoxazolyl.

A mammalian nicotine receptor partial agonist (NRPA), its optical isomers or a pharmaceutically acceptable salt of the forgoing compounds may be used in this invention. The term NRPA refers to chemical compounds that bind to neuronal nicotine receptor sites and elicit a partial agonist response.

The particular NRPA compounds listed above, which can be employed in the methods and pharmaceutical compositions of this invention, can be made by processes known in the chemical arts, for example, by the methods described in U.S. Pat. No. 6,235,734 by Pfizer Inc., issued May 22, 2001; WO 99/35131 by Pfizer Products Inc., published Jul. 15, 1999; and WO 99/55680 by Pfizer Products Inc., published Nov. 4, 1999; which are incorporated herein by reference.

Some of the NRPA compounds employed in this invention are ionizable at physiological conditions. Thus, for example some of the compounds of this invention are acidic and they form a salt with a pharmaceutically acceptable cation. The use of all such salts are within the scope of the pharmaceutical compositions and methods this invention and they can be prepared by conventional methods. For example, they can be prepared simply by contacting the acidic and basic entities, usually in a stoichiometric ratio, in either an aqueous, non-aqueous or partially aqueous medium, as appropriate. The salts are recovered either by filtration, by precipitation with a non-solvent followed by filtration, by evaporation of the solvent, or, in the case of aqueous solutions, by lyophilization, as appropriate.

In addition, some of the NRPA compounds employed in this invention are basic, and they form a salt with a pharmaceutically acceptable acid. All such salts are within the scope of this invention and they can be prepared by conventional methods. For example, they can be prepared simply by contacting the basic and acidic entities, usually in a stoichiometric ratio, in either an aqueous, non-aqueous or -partially aqueous medium, as appropriate. The salts are recovered either by filtration, by precipitation with a non-solvent followed by filtration, by evaporation of the solvent, or, in the case of aqueous solutions, by lyophilization, as appropriate.

The utility of the NRPA compounds employed in the present invention as medicinal agents in the treatment of ADHD mammals (e.g. humans) is demonstrated by the activity of the compounds of this invention in conventional assays and, in particular the assays described below. Such assays also provide a means whereby the activities of the compounds of this invention can be compared between them and with the activities of other known compounds. The results of these comparisons are useful for determining dosage levels in mammals, including humans, for the treatment of such diseases.

An estrogenic agent or a pharmaceutically acceptable form of estrogen may be used in this invention.

An estrogen receptor modulator agent or a SERM or a pharmaceutically acceptable salt of the foregoing compounds such as estrogen, lasofoxifene, droloxifene, tamoxifen, raloxifene (Evista) may be used in this invention.

Vitamin E or a pharmaceutically acceptable form of vitamin E may be used in this invention.

In general, the compounds of this invention can be made by processes which include processes known in the chemical arts, particularly in light of the description contained herein.

Some of the preparation methods useful for making the compounds of this invention may require protection of remote functionality (i.e., primary amine, secondary amine, carboxyl). The need for such protection will vary depending on the nature of the remote functionality and the conditions of the preparation methods. The need for such protection is readily determined by one skilled in the art The use of such protection/deprotection methods is also within the skill in the art For a general description of protecting groups and their use, see T. W. Greene, Protective Groups in Organic Synthesis, John Wiley & Sons, New York, 1991. The starting materials and reagents for the compounds of this invention are also readily available or can be easily synthesized by those skilled in the art using conventional methods of organic synthesis. For example, many of the compounds used herein are related to, or are derived from compounds found in nature, in which there is a large scientific interest and commercial need, and accordingly many such compounds are commercially available or are reported in the literature or are easily prepared from other commonly available substances by methods which are reported in the literature.

In certain situations, compounds of formula I may contain one or more asymmetric carbon atoms, so that the compounds can exist in different stereoisomeric forms. These compounds can be, for example, racemates or optically active forms. In these situations, the single enantiomers, i.e., optically active forms, can be obtained by asymmetric synthesis or by resolution of the racemates. Resolution of the racemates can be accomplished, for example, by conventional methods such as crystallization in the presence of a resolving agent, or chromatography, using for example a chiral HPLC column.

Some of the compounds of this invention are ionizable at physiological conditions. Thus, for example some of the compounds of this invention are acidic and they form a salt with a pharmaceutically acceptable cation. All such salts are within the scope of this invention and they can be prepared by conventional methods. For example, they can be prepared simply by contacting the acidic and basic entities, usually in a stoichiometric ratio, in either an aqueous, non-aqueous or partially aqueous medium, as appropriate. The salts are recovered either by filtration, by precipitation with a non-solvent followed by filtration, by evaporation of the solvent, or, in the case of aqueous solutions, by lyophilization, as appropriate.

In addition, some of the compounds of this invention are basic, and they form a salt with a pharmaceutically acceptable anion. All such salts are within the scope of this invention and they can be prepared by conventional methods. For example, they can be prepared simply by contacting the acidic and basic entities, usually in a stoichiometric ratio, in either an aqueous, non-aqueous or partially aqueous medium, as appropriate. The salts are recovered either by filtration, by precipitation with a non-solvent followed by filtration, by evaporation of the solvent, or, in the case of aqueous solutions, by lyophilization, as appropriate.

Non-toxic pharmaceutical salts include salts of acids such as hydrochloric, phosphoric, hydrobromic, sulfuric, sulfinic, formic, toluenesulfonic, methanesulfonic, nitric, benzoic, citric, tartaric, maleic, hydroiodic, alkanoic such as acetic, HOOC—(CH₂)ₙ—COOH where n is 0-4, and the like. Non-toxic pharmaceutical base addition salts include salts of bases such as sodium, potassium, calcium, ammonium, and the like. Those skilled in the art will recognize a wide variety of non-toxic pharmaceutically acceptable addition salts.

In addition, when the compounds of this invention form hydrates or solvates they are also within the scope of the invention.

The present invention also encompasses the acylated prodrugs of the compounds of formula I. Those skilled in the art will recognize various synthetic methodologies which may be employed to prepare non-toxic pharmaceutically acceptable addition salts and acylated prodrugs of the compounds encompassed by formula I.

The utility of the compounds of the present invention as medical agents in the treatment of conditions which present with low cognitive function (such as Alzheimer's Disease (AD), mild cognitive impairment, age-related cognitive decline, vascular dementia, Parkinson's disease, Huntington's disease, memory impairment associated with depresssion or anxiety, schizophrenia, Down's syndrome, stroke, traumatic brain injury (TBI), AIDS associated dementia and attention deficit disorder in mammals (e.g. humans) is demonstrated by the activity of the compounds of this invention in conventional assays and the in vitro assays described below. Cognitive function of the agents themselves or of the combination agents in mammals is measured in the radial arm maze in rodents or delayed matching to sample tests in primates. Such assays also provide a means whereby the activities of the compounds of this invention can be compared between themselves and with the activties of other known compounds. The results of these comparisons are useful for determining dosage levels in mammals, including humans, for the treatment of such diseases.

Biological Assays

Procedures

GABA_{A} Assays

The pharmaceutical utility of compounds and compositions of this invention is indicated by the following assays for GABA_{A} receptor activity.

Assays are carried out as described in Thomas and Tallman (J. Bio. Chem. 156: 9838-9842, J. Neurosci. 3: 433-440, 1983). Rat cortical tissue is dissected and homogenized in 25 volumes (w/v) of 0.05 M Tris HCl buffer (pH 7.4 at 4° C.). The tissue homogenate is centrifuged in the cold (4°) at 20,000×g for 20′. The supernatant is decanted and the pellet is rehomogenized in the same volume of buffer and again centrifuged at 20,000×g. The supernatant is decanted and the pellet is frozen at −20° C. overnight. The pellet is then thawed and rehomogenized in 25 volume (original wt/vol) of buffer and the procedure is carried out twice. The pellet is finally resuspended in 50 volumes. (w/vol of 0.05 M Tris HCl buffer (pH 7.4 at 40° C.).

Incubations contain 100 μl of tissue homogenate, 100 μl of radioligand 0.5 nM (³H-Ro15-1788 [³H-Flumazenil] specific activity 80 Ci/mmol), drug or blocker and buffer to a total volume of 500 μl. Incubations are carried out for 30 minutes at 4° C. then are rapidly filtered through GFB filters to separate free and bound ligand. Filters are washed twice with fresh 0.05 M Tris HCl buffer (pH 7.4 at 4° C.) and counted in a liquid scintillation counter. 1.0 mM diazepam is added to some tubes to determine nonspecific binding. Data are collected in triplicate determinations, averaged and % inhibition of total specific binding is calculated. Total Specific Binding=Total−Nonspecific. In some cases, the amounts of unlabeled drugs is varied and total displacement curves of binding are carried out. Data are converted to Ki's. Compounds of the invention when tested in the assay described above have Ki's of less than 1 μM.

In addition, the following assay may be used to determine if the compounds of the invention are agonists, antagonists, or inverse agonists, and, therefore, their specific pharmaceutical utility. The following assay can be employed to determine specific GABA_{A} receptor activity.

Assays are carried out as described in White and Gurley (NeuroReport 6: 1313-1316, 1995) and White, Gurley, Hartnett, Stirling, and Gregory (Receptors and Channels 3: 1-5, 1995) with modifications. Xenopus Laevis oocytes are enzymatcally isolated and injected with non-polyadenylated cRNA mixed in a ratio of 4:1:4 for human derived α, β, and γ subunits, respectively. For each subunit combination, sufficient message is injected to result in current amplitudes of >10 nA when 1 μM GABA is applied.

Electrophysiological recordings are carried out using the two electrode voltage-clamp technique at a membrane holding potential of −70 mV.

Compounds are evaluated against a GABA concentration that evokes <10% of the maximal evokable GABA current. Each oocyte is exposed to increasing concentrations of compound in order to evaluate a concentration/effect relationship. Compound efficacy is expressed as a percent-change in current amplitude: 100*((Ic/I)−1), where Ic is the GABA evoked current amplitude observed in the presence of compound and I is the GABA evoked current amplitude observed in the absence of compound.

Specificity of a compound for the Ro15-1788 site is determined following completion of the concentration/effect curve. After washing the oocyte sufficiently to remove previously applied compound, the oocyte is exposed to GABA+1 μM

Ro15-1788, followed by exposure to GABA+1 μM Ro15-1788+compound. Percent change due to addition of compound is calculated as described above. Any percent change observed in the presence of Ro15-1788 is subtracted from the percent changes in current amplitude observed in the absence of 1 μM Ro15-1788.

These net values are used for the calculation of average efficacy and EC₅₀ values.

To evaluate average efficacy and EC₅₀ values, the concentration/effect data are averaged across cells and fit to the logistic equation. Average values are reported as mean±standard error.

Nicotine Receptor Binding Assay

The effectiveness of the active compounds in suppressing nicotine binding to specific receptor sites is determined by the following procedure which is a modification of the methods of Lippiello, P. M. and Fernandes, K. G. (in The Binding of L-[³H]Nicotine To A Single Class of High-Affinity Sites in Rat Brain Membranes, Molecular Pharm., 29, 448-54, (1986)) and Anderson, D. J. and Americ, S. P. (in Nicotinic Receptor Binding of ³H-Cystisine, ³H-Nicotine and ³H-Methylcarmbamylcholine In Rat Brain, European J. Pharm., 253, 261-67 (1994)). Male Sprague-Dawley rats (200-300 g) from Charles River were housed in groups in hanging stainless steel wire cages and were maintained on a 12 hour light/dark cycle (7 a.m.-7 p.m. light period). They received standard Purina Rat Chow and water ad libitum. The rats were killed by decapitation. Brains were removed immediately following decapitation. Membranes were prepared from brain tissue according to the methods of Lippiello and Fernandez (Molec Pharmacol, 29, 448-454, (1986) with some modifications. Whole brains were removed, rinsed with ice-cold buffer, and homogenized at 0° in 10 volumes of buffer (w/v) using a Brinkmann Polytron™, setting 6, for 30 seconds. The buffer consisted of 50 mM Tris HCl at a pH of 7.5 at room temperature. The homogenate was sedimented by centrifugation (10 minutes; 50,000×g; 0° to 4° C.). The supernatant was poured off and the membranes were gently resuspended with the Polytron and centrifuged again (10 minutes; 50,000×g; 0 to 4° C. After the second centrifugation, the membranes were resuspended in assay buffer at a concentration of 1.0 g/100 mL. The composition of the standard assay buffer was 50 mM Tris HCl, 120 mM NaCl, 5 mM KCl, 2 mM MgCl₂, 2 mM CaCl₂ and has a pH of 7.4 at room temperature.

Routine assays were performed in borosilicate glass test tubes. The assay mixture typically consisted of 0.9 mg of membrane protein in a final incubation volume of 1.0 mL. Three sets of tubes were prepared wherein the tubes in each set contained 50 μL of vehicle, blank, or test compound solution, respectively. To each tube was added 200 μL of [³H]-nicotine in assay buffer followed by 750 μL of the membrane suspension. The final concentration of nicotine in each tube was 0.9 nM. The final concentration of cytisine in the blank was 1 μM. The vehicle consisted of deionized water containing 30 μL of 1 N acetic acid per 50 mL of water. The test compounds and cytisine were dissolved in vehicle. Assays were initiated by vortexing after addition of the membrane suspension to the tube. The samples were incubated at 0° to 4° C. in an iced shaking water bath. Incubations were terminated by rapid filtration under vacuum through Whatman GF/B™ glass fiber filters using a Brandel™ multi-manifold tissue harvester. Following the initial filtration of the assay mixture, filters were washed two times with ice-cold assay buffer (5 m each). The filters were then placed in counting vials and mixed vigorously with 20 ml of Ready Safe™ (Beckman) before quantification of radioactivity. Samples were counted in a LKB Wallach Rackbeta™ liquid scintillation counter at 40-50% efficiency. All determinations were in triplicate.

Calculations:

Specific binding (C) to the membrane is the difference between total binding in the samples containing vehicle only and membrane (A) and non-specific binding in the samples containing the membrane and cytisine (B), i.e.,

Specific binding=(C)=(A)−(B).

Specific binding in the presence of the test compound (E) is the difference between the total binding in the presence of the test compound (D) and non-specific binding (B), i.e., (E)=(D)−(B).

% Inhibition=(1−((E)/(C)) times 100.

The compounds of the invention that were tested in the above assay exhibited IC₅₀ values of less than 10 μM.

Dopamine Turnover

Rats were injected s.c. or p.o. (gavage) and then decapitated either 1 or 2 hours later. Nucleus accumbens was rapidly dissected (2 mm slices, 4° C., in 0.32 M sucrose), placed in 0.1 N perchloric acid, and then homogenized. After centrifugation. 10 uL of the supernatant was assayed by HPLC-ECD. Turnover/utilization of dopamine (DA) was calculated as the ratio of tissue concentrations of metabolites ([DOPAC]+[HVA]) to DA and expressed as percent of control.

Estrogen Receptor Binding Assay

cDNA Cloning of Human ERα and ERβ:

The coding region of human ERα_was cloned by RT-PCR from human breast cancer cell mRNA using Expand™ High Fidelity PCR System according to manufacturer's instructions (Boehringer-Mannheim, Indianapolis, Ind.). The coding region of human ERβ was cloned by RT-PCR from human testes and pituitary mRNA using Expand™ High Fidelity PCR System according to manufacturer's instructions (Boehringer-Mannheim, Indianapolis, Ind.). PCR products were cloned into pCR2.1 TA Cloning Kit (Invitrogen, Carlsbad, Calif.) and sequenced. Each receptor coding region was subcloned into the mammalian expression vector pcDNA3 ((Invitrogen, Carlsbad, Calif.).

Mammalian Cell Expression.

Receptor proteins were overexpressed in 293T cells. These cells, derived from HEK293 cells (ATCC, Manassas, Va.), have been engineered to stably express large T antigen and can therefore replicate plasmids containing a SV40 origin of replication to high copy numbers. 293T cells were transfected with either hERα-pcDNA3 or hERβ-pcDNA3 using lipofectamine as described by the manufacturer (Gibco/BRL, Bethesda, Md.). Cells were harvested in phosphate buffered saline (PBS) with 0.5 mM EDTA at 48 h post-transfection. Cell pellets were washed once with PBS/EDTA. Whole cell lysates were prepared by homogenization in TEG buffer (50 mM Tris pH 7.4, 1.5 mM EDTA, 50 mM NaCl, 10% glycerol, 5 mM DTT, 5 μg/ml aprotinin, 10 μg/ml leupeptin, 0.1 mg/ml Pefabloc) using a dounce homogenizer. Extracts were centrifuged at 100,000×g for 2 h at 4 C. and supernatants were collected. Total protein concentrations were determined using BioRad reagent (BioRad, Hercules, Calif.).

Competition Binding Assay.

The ability of various compounds to inhibit [³H]-estradiol binding was measured by a competition binding assay using dextrancoated charcoal as has been described (Leake R E, Habib F 1987 Steroic hormone receptors: assay and characterization. In: B. Green and R. E. Leake (eds). Steroid Hormones a Practical Approach. IRL Press Ltd, Oxford. 67-92.) 293T cell extracts expressing either hERα or hERβ were incubated in the presence of increasing concentrations of competitor and a fixed concentration of [³H]-estradiol (141 Ci/mmol, New England Nuclear, Boston, Mass.) in 50 mM Tris HCl pH 7.4, 1.5 mM EDTA, 50 mM NaCl, 10% glycerol, 5 mM DTT, 0.5 mg/mL β-lactoglobulin in a final volume of 0.2 mL. All competitors were dissolved in dimethylsulfoxide. The final concentration of receptor was 50 pM with 0.5 nM [³H]-estradiol. After 16 h at 4 C., dextran-coated charcoal (20 μL) was added. After 15 min at room temperature the charcoal was removed by centrifugation and the radioactive ligand present in the supernatant was measured by scintillation counting. All reagents were obtained from Sigma (St. Louis, Mo.) unless otherwise indicated.

Assays for Cognitive Dysfunction

Radial Arm Maze

Animals were food restricted to approximately 85% of their normal free-feeding weight and maintained at this level for 3 days prior to the first day of exposure to the maze.

Habituation: Reinforcement (Peanut Butter Chips) was placed near the entrance and at the mid-point of each arm, the animal was placed on the maze and allow to explore and consume the chips for a period of ten minutes, or until all chips were consumed. On the second day of habituation, the chips were placed at the midpoint and in a food cup at the end of each arm. Again, the animal was allowed ten minutes to explore or until all chips were consumed.

Training: Reinforcement is placed only in the food cup at the end of each arm. The animal is placed on the maze oriented away from the experimenter, and facing the same arm at the start of each trial. The timer is started and each entry is recorded in sequence. An entry is defined as all four paws entering the arm. The animal is allowed to choose until all eight arms are entered and the chip is consumed, or until 5 minutes has elapsed. Entry into an arm previously chosen is counted as an error. If an animal fails to choose all eight arms in 5 minutes, arms not chosen are also counted as errors. Animals are trained once a day. The criterion for learning is ≦1 error per day on at least two consecutive days. Dependent measures are number of errors, time to complete the maze, and number of days to reach criterion.

Drug Administration: Compounds to test for improvement of cognition are administered prior to each training session, or immediately following the training session.

Data analysis: Number of errors, and time to complete the maze are analyzed using repeated measures ANOVA Statview (SAS Institute) and post hoc testing using Dunnett's test.

Delayed Matching to Sample

Animals are tested in their home cages using a computer-automated training and testing system which measures and categorizes, in addition to percent correct at each delay, the latency of response at each step of each matching problem, and percent correct for every possible combination of matching stimuli (position and color). Stimuli on the test panels (attached to the home cages) are 2.54 cm diameter colored disks (red, yellow, or green) presented by light-emitting diodes located behind clear plastic push-keys. A trial is initiated with the illumination of the sample key by one of the colored disks. The sample light remains lit until the sample key is depressed by the subject, initiating one of four preprogrammed delay intervals, during which no disks are illuminated. Following the delay interval, two choice lights located below the sample key are illuminated. One of the choice lights matches the color of the sample light. These disks remain illuminated until the subject presses one of the two lighted keys. Key-presses of choice stimuli that match the color of the sample stimulus are rewarded by dispensing a 300 mg fruit-flavored food pellet. Non-matching choices are neither rewarded nor punished. Matching configurations are fully counterbalanced for side, delay, and color. A 5 sec inter-trial interval is used. Monkeys complete 96 trials on each day of testing. In standard DMTS sessions, four possible delay intervals between a subject's response to the sample light and the presentation of the two choice lights are employed: a Zero delay, and a Short, Medium, and Long delay. Short, Medium, and Long delay intervals are individually adjusted to produce stable performance levels approximating the following levels of accuracy: Zero delay (85-100% correct) Short (75-85% correct); Medium (65-75% correct); and Long (55-65% correct).

Variables relating to the monkey's performance are tabulated in a matrix for each daily session. It is possible to separate two main components of the DMTS task, a test of memory recall and a cognitive component, which tests the abstract conceptualization of “matching”. Baseline runs are generally performed on Mondays, with drug administered on Tuesdays and Thursdays. Wednesdays and Fridays the animals are tested, but no drug or vehicle will be administered. The animals are not run on weekends. We have not found any effect of day of testing on animal performance of the DMTS task. However, baseline performance is continuously monitored and redefined should the animal's performance change during the study. In such cases it would be necessary to determine if the baseline change is temporary (e.g., drug related) or permanent. In either case drug testing is discontinued to allow the adjustment(if necessary) of delay intervals until a typical and stable level of baseline performance is once again attained [Paule et al., Neurotoxicology and Teratology, 20, 493-502 (1998); Buccafusco et al., Behavioral Pharmacology, 10, 681-690 (1999)].

The combination of a GABA_{A} inverse agonist and a NRPA will result in increased efficacy in comparison to the efficacy displayed by either agent alone. In addition, such a combination may allow lower, subefficacious doses of each agent to be administered, resulting in efficacy similar to the one observed with higher doses of either agent alone and fewer side effects (or higher therapeutic index).

The combination of a GABA_{A} inverse agonist and estrogen and/or SERM will result in increased efficacy in comparison to the efficacy displayed by either agent alone. In addition, such a combination may allow lower, subefficacious doses of each agent to be administered, resulting in efficacy similar to the one observed with higher doses of either agent alone and fewer side effects (or higher therapeutic index).

The combination of a GABA_{A} inverse agonist and vitamin E will result in increased efficacy in comparison to the efficacy displayed by either agent alone. In addition, such a combination may allow lower, subefficacious doses of each agent to be administered, resulting in efficacy similar to the one observed with higher doses of either agent alone and fewer side effects (or higher therapeutic index).

The results of these comparisons are useful for determining dosage levels in mammals, including humans, for the treatment of such diseases.

Administration of the compositions of this invention can be via any method which delivers a compound of this invention systemically and/or locally. These methods include oral routes and transdermal routes, etc. Generally, the compounds of this invention are administered orally, but parenteral administration may be utilized (e.g., intravenous, intramuscular, subcutaneous or intramedullary). The two different compounds of this invention can be co-administered simultaneously or sequentially in any order, or a single pharmaceutical composition comprising a NRPA as described above and an anti-depressant or anxiolytic as described above in a pharmaceutically acceptable carrier can be administered.

The amount and timing of compounds administered will, of course, be based on the judgement of the prescribing physician. Thus, because of patient to patient variability, the dosages given below are a guideline and the physician may titrate doses of the agent to achieve the activity that the physician considers appropriate for the individual patient. In considering the degree of activity desired, the physician must balance a variety of factors such as cognitive function, age of the patient, presence of preexisting disease, as well as presence of other diseases (e.g., cardiovascular). The following paragraphs provide preferred dosage ranges for the various components of this invention (based on average human weight of 70 kg).

In general, an effective dosage for the GABA_{A} in the range of 0.001 to 30 mg/kg/day, preferably 0.01 to 10.0 mg/kg/day.

In general, an effective dosage for the NRPA in the range of 0.001 to 200 mg/kg/day, preferably 0.01 to 10.0 mg/kg/day.

The specific dosages for the estrogens or SERMS are as follows:

For estradiol the range is 0.005 to 0.03 mg/kg/day

For lasofoxifene the range is 0.0001 to 0.01 mg/kg/day

For droloxifene the range is 0.1 to 1.5 mg/kg/day

For tamoxifen the range is 0.05 to 0.5 mg/kg/day

For raloxifene (Evista) the range is 0.1 to 1.7 mg/kg/day

The specific dosages for vitamin E are 500-4,000 units a day, preferably 1,000 units once or twice a day.

It will be understood, however, that the specific dose level for any particular patient will depend up on a variety of factors including the activity of the specific compound employed, the age, body weight, general health, sex, diet, time of administration, route of administration, and rate of excretion, drug combination and the severity of the particular disease undergoing therapy.

The compositions of the present invention are generally administered in the form of a pharmaceutical composition comprising at least one of the compounds of this invention together with a pharmaceutically acceptable vehicle or diluent. Thus, the compounds of this invention can be administered individually or together in any conventional oral, parenteral or transdermal dosage form.

For oral administration a pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules; preferred materials in this connection also include lactose or milk sugar as well as high molecular weight polyethylene glycols. When aqueous suspensions and/or elixirs are desired for oral administration, the compounds of this invention can be combined with various sweetening agents, flavoring agents, coloring agents, emulsifying agents and/or suspending agents, as well as such diluents as water, ethanol, propylene glycol, glycerin and various like combinations thereof.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art.

For purposes of transdermal (e.g., topical) administration, dilute sterile, aqueous or partially aqueous solutions (usually in about 0.1% to 5% concentration), otherwise similar to the above parenteral solutions, are prepared.

Methods of preparing various pharmaceutical compositions with a certain amount of active ingredient are known, or will be apparent in light of this disclosure, to those skilled in this art. For examples, see Remington's Pharmaceutical Sciences, Mack Publishing Company, Easter, Pa., 15th Edition (1975).

Pharmaceutical compositions according to the invention may contain 0.1%-95% of the compound(s) of this invention, preferably 1%-70%. In any event, the composition or formulation to be administered will contain a quantity of a compound(s) according to the invention in an amount effective to treat the disease/condition of the subject being treated.

All patents, patent applications, and publications cited above are incorporated herein by reference in their entirety.

1. A pharmaceutical composition comprising a combination of an inverse agonist of the GABA_{A} α5 receptor subtype; a nicotine receptor partial agonist (NRPA), estrogen, selective estrogen modulators, or vitamin E; and a pharmaceutically acceptable carrier.

2. The pharmaceutical composition of claim 1, wherein the inverse agonist has a functional efficacy at the α5 receptor subtype of less than 20%, and a functional efficacy at the α₁, α₂ and α₃ receptor subtypes of between −20 and +20%.

3. A pharmaceutical composition comprising a combination of an inverse agonist of a GABA α1 and/or α5 receptor subtype; a nicotine receptor partial agonist (NRPA), estrogen, selective estrogen modulators, or vitamin E; and a pharmaceutically acceptable carrier; wherein the GABA_{A} inverse agonist has a functional efficacy at the α1 and/or α5 receptor subtypes of less than −5%, preferably less than −10%, and the efficacy measured at the α2 and α3 receptor subtypes is greater than 5% or preferably greater than 10%.

4. The pharmaceutical composition of claim 3, wherein the GABA_{A} inverse agonist has functional potency (EC50 values) at the α1 and/or α5 receptor subtypes of 200 nM, preferably less than 150 nM.

5. The pharmaceutical composition of claim 3, wherein the GABA_{A} inverse agonist has a functional efficacy at the α5 receptor subtype of less than −5%, preferably less than −10%, and the efficacy measured at the α1, α2 and α3 receptor subtypes is greater than 5% or preferably greater than 10%.

6. The pharmaceutical composition of claim 5 wherein the GABA_{A} inverse agonist has a functional potency (EC50 values) at the α5 receptor subtype of 200 nM, preferably less than 150 nM.

7. The pharmaceutical composition of claim 3 wherein the GABA_{A} inverse agonist at the α1 and/or α5 receptor subtypes has a binding Ki of 100 nM, preferably less than 30 nM.

8. The pharmaceutical composition of claim 1, wherein the GABA_{A} inverse agonist is selected from a compound of Formula I: 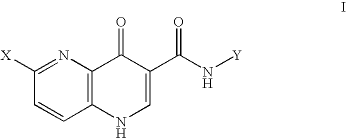
wherein:
X is hydrogen, halogen, —OR₁, NR₂R₃, C₁-C₆ alkyl optionally substituted with up to three groups selected independently from halogen and hydroxy, or —NR₂R₃; or
X is phenyl, naphthyl, 1-(5,6,7,8-tetrahydro)naphthyl or 4-(1,2-dihydro)indenyl, pyridinyl, pyrimidyl, isoquinolinyl, 1,2,3,4-tetrahydroisoquinolinyl, benzofuranyl, benzothienyl, each of which is optionally substituted with up to three groups selected from halogen, C₁-C₆ alkyl, C₁-C₄ alkoxy, C₁-C₆ alkylthio, hydroxy, amino, mono or di(C₁-C₆) alkylamino, cyano, nitro, trifluoromethyl; or
X represents a carbocyclic group (“the X carbocyclic group”) containing from 3-7 members, up to two of which are optionally hetero atoms selected from oxygen and nitrogen, where the X carbocyclic group is optionally substituted with one or more groups selected from halogen, (C₁-C₆)alkoxy, mono- or di(C₁-C₆)alkylamino, sulfonamide, aza(C₃-C₇)cycloalkyl, (C₃-C₇)cycloalkylthio, (C₁-C₆)alkylthio, phenylthio, or a heterocyclic group; and
Y is lower alkyl having 1-8 carbon atoms optionally substituted with up to two groups selected from halogen, (C₁-C₆)alkoxy, mono- or di(C₁-C₆)alkylamino, sulfonamide, aza(C₃-C₇)cycloalkyl, (C₃-C₇)cycloalkylthio, (C₁-C₆)alkylthio, phenylthio, a heterocyclic group, —OR₄, —NR₅R₆, SR₇, or aryl; or
Y is a carbocyclic group (“the Y carbocyclic group”) having from 3-7 members atoms, where up to three of which are optionally hetero atoms selected from oxygen and nitrogen and where any member of the Y carbocyclic group is optionally substituted with halogen, —OR₄, —NR₅R₆, SR₇, aryl or a heterocyclic group; and
R₁ is hydrogen, lower alkyl having 1-6 carbon atoms, or cycloalkyl having 3-7 carbon atoms, where each alkyl may be optionally substituted with —OR₄ or —NR₅R₆;
R₂ and R₃ are the same or different and represent hydrogen, lower alkyl optionally mono- or disubstituted with alkyl, aryl, halogen, or mono- or di-lower alkyl; aryl or aryl (C₁-C₆)alkyl where each aryl is optionally substituted with up to three groups selected from halogen, hydroxy, C₁-C₆ alkyl, C₁-C₆ alkoxy, or mono- or di(C₁-C₆)alkylamino;
cycloalkyl having 3-7 carbon atoms optionally mono or disubstituted with halogen, alkoxy, or mono- or di-lower alkyl; or
—SO₂R₈;
R₄ is as defined for R₁;
R₅ and R₆ carry the same definitions as R₂ and R₃, respectively;
R₇ is hydrogen, lower alkyl having 1-6 carbon atoms, or cycloalkyl having 3-7 atoms; and
R₈ is lower alkyl having 1-6 carbon atoms, cycloalkyl having 3-7 carbon atoms, or optionally substituted phenyl;
or an isomer or hydrate thereof, or a pharmaceutically acceptable salt thereof.

9. The pharmaceutical composition of claim 1, wherein the GABA_{A} inverse agonist is selected from the group consisting of:
N-n-Butyl-6-chloro-4-oxo-1,4-tetrahydro-1,5-naphthyridine-3-carboxamide;
N-n-Butyl-6-ethoxy-4-oxo-1,4-tetrahydro-1,5-naphthyridine-3-carboxamide;
N-(2-Ethylthio)ethyl-6-methoxy-4-oxo-1,4-tetrahydro-1,5-naphthyridine-3-carboxamide;
N-n-Pentyl-6-ethoxy-4-oxo-1,4-tetrahydro-1,5-naphthyridine-3-carboxamide;
N-Benzyl-6-ethoxy-4-oxo-1,4-tetrahydro-1,5-naphthyridine-3-carboxamide;
N-(2-Tetrahydrofuranyl)methyl-6-ethoxy-4-oxo-1,4-tetrahydro-1,5-naphthyridine-3-carboxamide;
N-Isoamyl-6-ethoxy-4-oxo-1,4-tetrahydro-1,5-naphthyridine-3 carboxamide;
N-(3-Methoxybenzyl)-6-ethoxy-4-oxo-1,4-tetrahydro-1,5-naphthyridine-3-carboxamide;
N-(3-Ethoxy)propyl-6-ethoxy-4-oxo-1,4-tetrahydro-1,5-naphthyridine-3-carboxamide;
N-2-(2-Methyl)butyl-4-ethoxy-4-oxo-1,4-tetrahydro-1,5-naphthyridine-3 carboxamide;
N-5-Pentanol-6-ethoxy-4-oxo-1,4-tetrahydro-1,5-naphthyridine-3-carboxamide;
N-Benzyl-6-methoxy-4-oxo-1,4-tetrahydro-1,5-naphthyridine-3-carboxamide;
N-(2-Fluorobenzyl)-6-methoxy-4-oxo-1,4-tetrahydro-1,5-naphthyridine-3-carboxamide;
N-(3-Fluorobenzyl)-6-methoxy-4-oxo-1,4-tetrahydro-1,5-naphthyridine-3 carboxamide;
N-(4-Fluorobenzyl)-6-methoxy-4-oxo-1,4-tetrahydro-1,5-naphthyridine-3-carboxamide;
N-(4/5-Imidazolyl)methyl-6-ethoxy-4-oxo-1,4-tetrahydro-1,5-naphthyridine-3-carboxamide;
N-(3-Thienyl)methyl-6-ethoxy-4-oxo-1,4-tetrahydro-1,5-naphthyridine-3-carboxamide;
N-(2-Tetrahydropyranyl)methyl-6-ethoxy-4-oxo-1,4-tetrahydro-1,5-naphthyridine-3-carboxamide;
N-(2-Fluorobenzyl)-6-ethoxy-4-oxo-1,4-tetrahydro-1,5-naphthyridine-3-carboxamide;
N-(3,5-Fluorobenzyl)6-ethoxy-4-oxo-1,4-tetrahydro-1,5-naphthyridine-3-carboxamide;
N-(4-Fluorobenzyl)-6-ethoxy-4-oxo-1,4-tetrahydro-1,5-naphthyridine-3-carboxamide;
N-(4-Methoxybenzyl)-6-ethoxy-4-oxo-1,4-tetrahydro-1,5-naphthyrdine-3-carboxamide;
N-(4-Methylbenzyl)-6-ethoxy-4-oxo-1,4-tetrahydro-1,5-naphthyridine-3-carboxamide;
N-(2-Thienyl)methyl-6-(2-methoxyethoxy)-4-oxo-1,4-tetrahydro-1,5-naphthyridine-3-carboxamide;
N-(2-Thienyl)methyl-6-morpholino-4-oxo-1,4-tetrahydro-1,5-naphthyridine-3-carboxamide;
N-(2-Thienyl)methyl-6-dimethylamino-4-oxo-1,4-tetrahydro-1,5-naphthyridine-3-carboxamide;
N-(4-Methylaminomethyl)benzyl-6-ethoxy-4-oxo-1,4-tetrahydro-1,5-naphthyridine-3-carboxamide;
N-(3-Methylaminomethyl)benzyl-6-ethoxy-4-oxo-1,4-tetrahydro 1,5 naphthyridine-3-carboxamide hydrochloride; and
N-[4-(Imidazolylmethy)lbenzyl-6-ethoxy-4-oxo-1,4-tetrahydro-1,5-naphthyridine-3-carboxamide.

10. The pharmaceutical composition of claim 1 in which the NRPA is selected from the group consisting of:
9-bromo-1,2,3,4,5,6-hexahydro-1,5-methano-pyrido[1,2-a][1,5]diazocin-8-one;
9-chloro-1,2,3,4,5,6-hexahydro-1,5-methano-pyrido[1,2-a][1,5]diazocin-8-one;
9-flouro-1,2,3,4,5,6-hexahydro-1,5-methano-pyrido[1,2-a][1,5]diazocin-8-one;
9-ethyl-1,2,3,4,5,6-hexahydro-1,5-methano-pyrido[1,2-a][1,5]diazocin-8-one;
9-methyl-1,2,3,4,5,6-hexahydro-1,5-methano-pyrido[1,2-a][1,5]diazocin-8-one;
9-phenyl-1,2,3,4,5,6-hexahydro-1,5-methano-pyrido[1,2-a][1,5]diazocin-8-one;
9-vinyl-1,2,3,4,5,6-hexahydro-1,5-methano-pyrido[1,2-a][1,5]diazocin-8-one;
9-bromo-3-methyl-1,2,3,4,5,6-hexahydro-1,5-methano-pyrido[1,2a][1,5]diazocin-8-one;
3-benzyl-9-bromo-1,2,3,4,5,6-hexahydro-1,5-methano-pyrido[1,2-a][1,5]diazocin-8-one;
3-benzyl-9-chloro-1,2,3,4,5,6-hexahydro-1,5-methano-pyrido[1,2-a][1,5]diazodin-8-one;
9-acetyl-1,2,3,4,5,6-hexahydro-1,5-methano-pyrido[1,2a][1,5]diazocin-8-one;
9-iodo-1,2,3,4,5,6-hexahydro-1,5-methano-pyrido[1,2a][1,5]diazocin-8-one;
9-cyano-1,2,3,4,5,6-hexahydro-1,5-methano-pyrido[1,2a][1,5]diazocin-8-one;
9-ethynyl-1,2,3,4,5,6-hexahydro-1,5-methano-pyrido[1,2a][1,5]diazocin-8-one;
9-(2-propenyl)-1,2,3,4,5,6-hexahydro-1,5-methano-pyrido[1,2a][1,5]diazocin-8-one;
9-(2-propyl)1,2,3,4,5,6-hexahydro-1,5-methano-pyrido[1,2a][1,5]diazocin-8-one;
9-carbomethoxy-1,2,3,4,5,6-hexahydro-1,5-methano-pyrido[1,2a][1,5]diazocin-8-one;
9-carboxyaldehyde-1,2,3,4,5,6-hexahydro-1,5-methano-pyrido[1,2a][1,5]diazocin-8-one;
9-(2,6-difluorophenyl 1,2,3,4,5,6-hexahydro-1,5-methano-pyrido[1,2a][1,5]diazocin-8-one;
9-phenyl-1,2,3,4,5,6-hexahydro-1,5-methano-pyrido[1,2a][1,5]diazocin-8-one;
9-(2-fluorophenyl)-1,2,3,4,5,6-hexahydro-1,5-methano-pyrido[1,2a][1,5]diazocin-8-one;
9-(4-fluorophenyl 1,2,3,4,5,6-hexahydro-1,5-methan-pyrido[1,2a][1,5]diazocin-8-one;
9-(3-fluorophenyl)-1,2,3,4,5,6-hexahydro-1,5-methano-pyrido[1,2a][1,5]diazocin-8-one;
9-(3,5-difluorophenyl)-1,2,3,4,5,6-hexahydro-1,5-methano-pyrido[1,2a][1,5]diazocin-8-one;
9-(2,4-difluorophenyl)-1,2,3,4,5,6-hexahydro-1,5-methano-pyrido[1,2a][1,5]diazocin-8-one;
9-(2,5-difluorophenyl)-1,2,3,4,5,6-hexahydro-1,5-methano-pyrido[1,2a][1,5]diazocin-8-one;
6-methyl-5-oxo-6,13-diazatetracyclo[9.3.1.0^{2,10}.0^{4,8}]pentadeca-2(10),3,8-triene;
5-oxo-6,3-diazatetracyclo[9.3.1.0^{2,10}.0^{4,8}]pentadeca-2(10),3,8-triene;
6-oxo-5,7,13-triazatetracyclo[9.3.1.0^{2,10}.0^{4,8}]pentadeca-2(10),3,8-triene;
4,5-difluoro-10-aza-tricyclo[6.3.1.0^{2,7}]dodeca-2(7),3,5-triene;
5-fluoro-10-aza-tricyclo[6.3.1.0^{2,7}]dodeca-2(7),3,5-triene-4-carbonitrile;
4-ethynyl-5-fluoro-10-aza-tricyclo[6.3.1.0^{2,7}]dodeca-2(7),3,5-triene;
5-ethynyl-10-aza-tricyclo[6.3.1.0^{2,7}]dodeca-2(7),3,5-triene carbonitrile;
6-methyl-5-thia-5-dioxa-6,13-diatetracyclo[9.3.1.0^{2,10}.0^{4,8}]pentadeca-2(10),3,8-triene;
10-aza-tricyclo[6.3.1.0^{2,7}]dodeca-2(7),3,5-triene;
4-fluoro-10-aza-tricyclo[6.3.1.0^{2,7}]dodeca-2(7),3,5-triene;
4-methyl-10-aza-tricyclo[6.3.1.0^{2,7}]dodeca-2(7),3,5-triene;
4-trifluoromethyl-10-aza-tricyclo[6.3.1.0^{2,7}]dodeca-2(7),3,5-triene;
4-nitro-10-azatricyclo[6.3.1.0^{2,7}]dodeca-2(7),3,5-triene;
7-methyl-5,7,13-triazatetracyclo[9.3.1.0^{2,10}.0^{4,8}]pentadeca-2(10),3,5,8-tetraene;
6-methyl-5,7,13-triazatetracyclo[9.3.1.0^{2,10}.0^{4,8}]pentadeca-2(10),3,5,8-tetraene;
6,7-dimethyl-5,7,13-triazatetracyclo[9.3.1.0^{2,10}.0^{4,8}]pentadeca-2(10),3,5,8-tetraene;
6-methyl-7-phenyl-5,7,13-triazatetracyclo[9.3.1.0^{2,10}.0^{4,8}]pentadeca-2(10),3,5,8-tetraene;
6,7-dimethyl-5,8,14-triazatetracyclo[10.3.1.0^{2,11}.0^{4,9}]hexadeca-2(11),3,5,7,9-pentaene;
5,8,14-triazatetracyclo[10.3.1.0^{2,11}.0^{4,9}]hexadeca-2(11),3,5,7,9-pentaene;
14-methyl-5,8,14-triazatetracyclo[10.3.1.0^{2,11}. 0^{4,8}]hexadeca-2(11),3,5,7,9-pentaene;
5-oxa-7,13-diazatetracyclo[9.3.1.0^{2,10}.0^{4,8}]pentadeca-2(10),3,6,8-tetraene;
6-methyl-5-oxa-7,13-diazatetracyclo[9.3.1.0^{2,10}^{4,8}]pentadeca-2(10),3,6,8-tetraene;
4-chloro-10-azatricyclo[6.3.1.0^{2,7}]dodeca-2(7),3,5-triene;
10-azatricyclo[6.3.1.0^{2,7}]dodeca-2(7),3,5-trien-4-yl cyanide;
1-(10-azatricyclo[6.3.1.0^{2,7}]dodeca-2(7),3,5-trien-4-yl)1-ethanone;
10-azatricyclo[6.3.1.0^{2,7}]dodeca-2(7),3,5-trien-4-ol;
7-methyl-5-oxa-6,13-diazatetracyclo[9.3.1.0^{2,10}.0^{4,8}]pentadeca-2,4(8),6,9-tetraene;
4,5-dichloro-10-azatricyclo[6.3.1.0^{2,7}]dodeca-2(7),3,5-triene;
11-azatricyclo[7.3.1.0^{2,7}]trideca-2(7),3,5-triene-5-carbonitrile;
1-[11-azatricyclo[7.3.1.0^{2,7}]trideca-2(7),3,5-trien-5-yl]-1-ethanone;
1-[11-azatricyclo[7.3.1.0^{2,7}]trideca-2(7),3,5-trien-5-yl]-1-propanone;
4-fluoro-11-azatricyclo[7.3.1.0^{2,7}]trideca-2(7),3,5-triene-5-carbonitrile;
5-fluoro-11-azatricyclo[7.3.1.0^{2,7}]trideca-2(7),3,5-triene carbonitrile;
6-methyl-7-thia-5,14-diazatetracyclo[10.3.1.0^{2,10}.0^{4,8}]hexadeca-2(10),3,5,8-tetraene;
6-methyl-5,7,14-triaztetracyclo[10.3.1.0^{2,10}.0^{4,8}]hexadeca-2(10),3,5,8-tetraene;
6,7-dimethyl-5,7,14-triazatetracyclo[10.3.1.0^{2,10}.0^{4,8}]hexadeca-2(10),3,5,8-tetraene;
5,7,14-triazatetracyclo[10.3.1.0^{2,10}.0^{4,8}]hexadeca-2(10),3,5,8-tetraene;
5,6-dimethyl-5,7,14-triazatetracyclo[10.3.1.0^{2,10}.0^{4,8}]hexadeca-2(10),3,6,8-tetraene;
5-methyl-5,7,14-triazatetracyclo[10.3.1.0^{2,10}.0^{4,8}]hexadeca-2(10),3,6,8-tetraene;
6-(trifluoromethyl)7-thia-5,14-diazatetracyclo[10.3.1.0^{2,10}.0^{4,8}]hexadeca-2(10),3,5,8-tetraene;
5,8,15-triazatetracyclo[11.3.1.0^{2,11}.0^{4,9}]heptadeca-2(11),3,5,7,9-pentaene;
7-methyl-5,8,15-triazatetracyclo[11.3.1.0^{2,11}.0^{4,9}]heptadeca-2(11),3,5,7,9-pentaene;
6-methyl-5,8,15-triazatetracyclo[11.3.1.0^{2,11}.0^{4,9}]heptadeca-2(11),3,5,7,9 pentaene;
6,7-dimethyl-5,8,15-triazatetracyclo[11.3.1.0^{2,11}.0^{4,9}]heptadeca-2(11),3,5,7,9-pentaene;
7-oxa-5,14-diazatetracyclo[10.3.10^{2,10}.0^{4,8}]hexadeca-2(10),3,5,8-tetraene;
6-methyl-7-oxa-5,14-diazatetracyclo[10.3.1.0^{2,10}.0^{4,8}]hexadeca-2(10),3,5,8-tetraene;
5-methyl-7-oxa-6,14-diazatetracyclo[10.3.1.0^{2,10}.0^{4,8}]hexadeca-2(10),3,5,8-tetraene;
6-methyl-5-oxa-7,14-diazatetracyclo[10.3.1.0^{2,10}.0^{4,8}]hexadeca-2(10),3,6,8-tetraene;
7-methyl-5-oxa-6,14-diazatetracyclo[10.3.1.0^{2,10}.0^{4,8}]hexadeca-2(10),3,6,8-tetraene;
4,5-difluoro-11-azatricyclo[7.3.1.0^{2,7}]trideca-2(7),3,5-triene;
4-chloro-5-fluoro-11-azatricyclo[7.3.1.0^{2,7}]trideca-2(7),3,5-triene;
5-chloro-4-fluoro-11-azatricyclo[7.3.1.0^{2,7}]trideca-2(7),3,5-triene;
4-(1-ethynyl)-5-fluoro-11-azatricyclo[7.3.1.0^{2,7}]trideca-2(7),3,5-triene;
5-(1-ethynyl)-4-fluoro-11-azatricyclo[7.3.1.0^{2,7}]trideca-2(7),3,5-triene;
5,6-difluoro-11-aza-tricyclo[7.3.1.0^{2,7}]trideca-2,4,6-triene;
6-trifluoromethyl-11-aza-tricyclo[7.3.1.0^{2,7}]trideca-2,4,6-triene;
6-methoxy-11-aza-tricyclo[7.3.1.0^{2,7}]trideca-2(7),3,5-triene;
11-aza-tricyclo[7.3.1.0^{2,7}]trideca-2(7),3,5-trien-6-ol;
6-fluoro-11-aza-tricyclo[7.3.1.0^{2,7}]trideca-2(7),3,5-triene;
11-aza-tricyclo[7.3.1.0^{2,7}]trideca-2(7),3,5-trien-5-ol;
4-nitro-11-aza-tricyclo[7.3.1.0^{2,7}]trideca-2(7),3,5-triene;
5-nitro-11-aza-tricyclo[7.3.1.0^{2,7}]trideca-2(7),3,5-triene;
5-fluoro-11-aza-tricyclo[7.3.1.0^{2,7}]trideca-2(7),3,5-triene; and
6-hydroxy-5-methoxy-11-aza-tricyclo[7.3.1.0^{2,7}]trideca-2(7),3,5-triene and their pharmaceutically acceptable salts and their optical isomers.

11. The pharmaceutical composition of claim 1, in which the NRPA is selected from the group consisting of:
9-bromo-1,2,3,4,5,6-hexahydro-1,5-methano-pyrido[1,2-a][1,5]diazocin-8-one;
9-chloro-1,2,3,4,5,6-hexahydro-1,5-methano-pyrido[1,2-a][1,5]diazocin-8-one;
9-flouro-1,2,3,4,5,6-hexahydro-1,5-methano-pyrido[1,2-a][1,5]diazocin-8-one;
9-acetyl-1,2,3,4,5,6-hexahydro-1,5-methano-pyrido[1,2-a][1,5]diazocin-8-one;
9-iodo-1,2,3,4,5,6-hexahydro-1,5-methano-pyrido[1,2-a][1,5]diazocin-8-one;
9-cyano-1,2,3,4,5,6-hexahydro-1,5-methano-pyrido[1,2-a][1,5]diazocin-8-one;
9-carbomethoxy-1,2,3,4,5,6-hexahydro-1,5-methano-pyrido[1,2a][1,5]diazocin-8-one;
9-carboxyaldehyde-1,2,3,4,5,6-hexahydro-1,5-methano-pyrido[1,2a][1,5]diazocin-8-one;
9-(2,6-difluorophenyl)-1,2,3,4,5,6-hexahydro-1,5-methano-pyrido[1,2a][1,5]diazocin-8-one;
9-phenyl-1,2,3,4,5,6-hexahydro-1,5-methano-pyrido[1,2a][1,5]diazocin-8-one;
9-(2-fluorophenyl)-1,2,3,4,5,6-hexahydro-1,5-methano-pyrido[1,2a][1,5]diazocin-8-one;
6-methyl-5-thia-5-dioxa-6,13-diazatetracyclo[9.3.1.0^{2,10}.0^{4,8}]pentadeca-2(10),3,8-triene;
4-fluoro-10-aza-tricyclo[6.3.1.0^{2,7}]dodeca-2(7),3,5-triene;
4-trifluoromethyl-10-aza-tricyclo[6.3.1.0^{2,7}]dodeca-2(7),3,5-triene;
4-nitro-10-azatricyclo[6.3.1.0^{2,7}]dodeca-2(7),3,5-triene;
6-methyl-5,7,13-triazatetracyclo[9.3.1.0^{2,10}.0^{4,8}]pentadeca-2(10),3,5,8-tetraene;
6,7-dimethyl-5,8,14-triazatetracyclo[10.3.1.0^{2,11}.0^{4,9}]hexadeca-2(11),3,5,7,9-pentaene;
5,8,14-triazatetracyclo[10.3.1.0^{2,11}.0^{4,9}]hexadeca-2(11),3,5,7,9-pentaene;
5-oxa-7,13-diazatetracyclo[9.3.1.0^{2,10}.0^{4,8}]pentadeca-2(10),3,6,8-tetraene;
6-methyl-5-oxa-7,13-diazatetracyclo[9.3.1.0^{2,10}.0^{4,8}]pentadeca-2(10),3,6,8-tetraene;
10-azatricyclo[6.3.1.0^{2,7}]dodeca-2(7),3,5-trien-4-yl cyanide;
1-(10-azatricyclo[6.3.1.0^{2,7}]dodeca-2(7),3,5-trien-4-yl)-1-ethanone;
11-azatricyclo[7.3.1.0^{2,7}]trideca-2(7),3,5-triene-5-carbonitrile;
1-[11-azatricyclo[7.3.1.0^{2,7}]trideca-2(7),3,5-trien-5-yl]-1-ethanone;
1-[11-azatricyclo[7.3.1.0^{2,7}]trideca-2(7),3,5-trien-5-yl]-1-propanone;
4-fluoro-11-azatricyclo[7.3.1.0^{2,7}]trideca-2(7),3,5-triene-5-carbonitrile;
5-fluoro-11-azatricyclo[7.3.1.0^{2,7}]trideca-2(7),3,5-triene-4-carbonitrile;
6-methyl-7-thia-5,14-diazatetracyclo[10.3.1.0^{2,10}.0^{4,8}]hexadeca-2(10),3,5,8-tetraene;
6-methyl-5,7,14-triazatetracyclo[10.3.1.0^{2,10}.0^{4,8}]hexadeca-2(10),3,5,8-tetraene;
6,7-dimethyl-5,7,14-triazatetracyclo[10.3.1.0^{2,10}.0^{4,8}]hexadeca-2(10),3,5,8-tetraene;
6-methyl-7-oxa-5,14-diazatetracyclo[10.3.1.0^{2,10}.0^{4,8}]hexadeca-2(10),3,5,8-tetraene;
6-methyl-5-oxa-7,14-diazatetracyclo[10.3.1.0^{2,10}.0^{4,8}]hexadeca-2(10),3,6,8-tetraene;
5,6-difluoro-11-aza-tricyclo[7.3.1.0^{2,7}]trideca-2,4,6-triene;
6-trifluoromethyl-11-aza-tricyclo[7.3.1.0^{2,7}]trideca-2,4,6-triene;
6-methoxy-11-aza-tricyclo[7.3.1.0^{2,7}]trideca-2(7),3,5-triene;
6-fluoro-11-aza-tricyclo[7.3.1.0^{2,7}]trideca-2(7),3,5-triene; and
11-aza-tricyclo[7.3.1.0^{2,7}]trideca-2(7),3,5-trien-5-ol and their pharmaceutically acceptable salts and their optical isomers.

12. The pharmaceutical composition of claim 1, wherein the GABA_{A} inverse agonist is N-Benzyl-6-ethoxy-4-oxo-1,4-tetrahydro-1,5-naphthyridine-3-carboxamide, or a prodrug thereof, or a pharmaceutically acceptable salt or solvate of said compound or prodrug.

13. A method for treating a cognitive disorder in a mammal, comprising administering to a mammal in need of said treatment an effective amount of a combination comprising a GABA_{A} α5 receptor subtype; and a nicotine receptor partial agonist (NRPA), estrogen, selective estrogen modulators, or vitamin E.

14. The method of claim 13, wherein the a GABA_{A} α5 receptor subtype and the NRPA are administered simultaneously.

15. The method of claim 13, wherein the a GABA_{A} α5 receptor subtype and the NRPA are administered sequentially.

16. The method of claim 13, wherein the GABA_{A} inverse agonist is N-Benzyl-6-ethoxy-4-oxo-1,4-tetrahydro-1,5-naphthyridine-3-carboxamide, or a prodrug thereof, or a pharmaceutically acceptable salt or solvate of said compound or prodrug.
